SEQUENCE LISTING (10 sequences)

SEQ ID NO: 1 - DNA, 438 bp, Mus musculus
atttttggtatctttgatgacaataatgggggattttgaaagcttagctttaaatttcttttaattaaaaaaaaatgctaggcagaatgactcaaattacgttggatacaattgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtagcagagaggaaccattgccaccttcagattttcctgtaacgatcgggaactggcatcttcagggagtagcttaggtcagt

SEQ ID NO: 2 - DNA, 220 bp, Mus musculus
attcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaactt

SEQ ID NO: 3 - DNA, 27 bp, Artificial Sequence
  A synthetic oligonucleotide
gtatctttgatgacaataatgggggat

SEQ ID NO: 4 - DNA, 27 bp, Artificial Sequence
  A synthetic oligonucleotide
gtatctttgatgacaataatgggggat

SEQ ID NO: 5 - DNA, 27 bp, Artificial Sequence
  A synthetic oligonucleotide
ggcagaatgactcaaattacgttggat

SEQ ID NO: 6 - DNA, 27 bp, Artificial Sequence
  A synthetic oligonucleotide
ttcaactgtatccaacgtaatttgagt

SEQ ID NO: 7 - DNA, 27 bp, Artificial Sequence
  A synthetic oligonucleotide
gatcgggaactggcatcttcagggagt

SEQ ID NO: 8-9 - skipped (2 entries)

SEQ ID NO: 10 - protein, 528 aa, Mus musculus
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLKSILDTLDVMAYNKLNVFHWHLVDDQSFPYESFTFPELMRKGSYSLSHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGQDWRKFYKVEPLAFEGTPEQKALVIGGEACMWGEYVDATNLVPRLWPRAGAVAERLWSNKLTRDMDDAYDRLSHFRCELVRRGVAAQPLYAGYCNQEFEQT

(19) United States
(12) Patent Application Publication
(10) Pub. No.: US 2023/0201373 A1
(43) Pub. Date: Jun. 29, 2023

(54) CRISPR-MEDIATED GENOME EDITING WITH VECTORS

(71) Applicant: Regents of the University of Minnesota, Minneapolis, MN (US)

(72) Inventors: Li Ou, Eden Prairie, MN (US); Chester B. Whitley, Brooklyn Park, MN (US); Michael Przybilla, Chaska, MN (US)

(21) Appl. No.: 16/954,171

(22) PCT Filed: Dec. 14, 2018
(86) PCT No.: PCT/US2018/065747

§ 371 Date: Jun. 15, 2020

Related U.S. Application Data
(60) Provisional application No. 62/599,642, filed on Dec. 15, 2017.

(51) Int. Cl. A61K 48/00; C12N 15/11; C12N 9/22; C12N 15/90; C12N 15/86; A61K 38/46; A61K 31/7088; A61K 38/47; A61P 25/00

(52) U.S. Cl. CPC A61K 48/0066; A61K 31/7088; A61K 38/47; A61K 38/465; A61P 25/00; C12N 9/22; C12N 15/11; C12N 15/86; C12N 15/907; C12N 2310/20; C12N 2750/14143; C12N 2750/14171; C12N 2800/80

(57) ABSTRACT

Compositions and methods for Cas-based ex vivo and in vivo gene therapy applications are provided.

### CROSS-REFERENCE TO RELATED APPLICATIONS

This application claims the benefit of the filing date of U.S. application No. 62/599,642, filed on Dec. 15, 2017, the disclosure of which is incorporated by reference herein.

### BACKGROUND

Gene therapy holds enormous potential for a new era of human therapeutics. These methodologies will allow treatment for conditions that heretofore have not been addressable by standard medical practice. One area that is especially promising is the ability to add a transgene to a cell to cause that cell to express a product that previously not being produced (or produced at insufficient levels) in that cell. Examples of uses of this technology include the insertion of a gene encoding a therapeutic protein, insertion of a coding sequence encoding a protein that is somehow lacking in the cell or in the individual and insertion of a sequence that encodes a structural nucleic acid such as a microRNA or siRNA.

Transgenes can be delivered to a cell by a variety of ways, such that the transgene becomes integrated into the cell’s own genome and is maintained there. In recent years, a strategy for transgene integration has been developed that uses cleavage with site-specific nucleases for targeted insertion into a chosen genomic locus (see, e.g., U.S. Pat. No. 7,888,121). Nucleases, such as zinc finger nucleases (ZFNs), transcription activator-like effector nucleases (TALENs), or nuclease systems such as the CRISPR/Cas system (utilizing an engineered guide RNA), are specific for targeted genes and can be utilized such that the transgene construct is inserted by either homology directed repair (HDR) or by end capture during non-homologous end joining (NHEJ) driven processes.

### SUMMARY

in one embodiment, the invention provides for delivery of one or more genes encoding proteins using CRISPR/Cas, delivered via one or more vectors such as plasmids or viral vectors, including but not limited to lentivirus vectors, adenovirus vectors, adeno-associated virus (AAV) vectors, e.g., AAV2, AAV5, AAV6, AAV8, or AAV9, or herpesvirus vectors, which proteins may be useful to prevent, inhibit or treat diseases such as monogenic diseases, e.g., lysosomal storage diseases, hemophilia, thalassemia, sickle cell diseases and the like. In one embodiment, at least one or two vectors are used to deliver one or more CRISPR components, e.g., nucleic acid encoding Cas, gRNA(s), a gene encoding the protein or interest, e.g., which is optionally promoterless, for targeted insertion into the genome of a host cell, e.g., ex vivo or in vivo. In one embodiment, systemic of the one or more vectors administration is employed. In one embodiment, Cas may be supplied in trans. Combinations of different vectors and/or proteins may be used. Sequences for gRNA and homology arms flanking the gene of interest may be directed to any insertion (target) site in the genome of a host cell so long as the site allows for adequate expression of the introduced gene. Exemplary insertion sites include but are not limited to the albumin locus, AAVS1, Rosa26, CCR5, HPRT, and the alpha fetoprotein locus. In one embodiment, exemplary host genome sites for insertion have few if any polymorphisms. In one embodiment, the vector(s) is/are mRNA, e.g., in a nanoparticle such as a liposome. In one embodiment, the vector(s) is/are plasmid vectors, e.g., in a nanoparticle such as a liposome. In one embodiment, the vector(s) is/are viral vectors. In one embodiment, one vector is employed. In one embodiment, two vectors are employed.

in one embodiment, a method to prevent, inhibit or treat a disease in a mammal or a mammalian cell is provided. The method includes administering an effective amount of i) Cas or an isolated nucleic encoding Cas, e.g., a vector comprising an isolated nucleic encoding Cas, and ii) isolated nucleic acid for one or more gRNAs comprising a targeting sequence for a genomic target and nucleic acid comprising a coding sequence for a prophylactic or therapeutic gene product flanked by homology arms, e.g., a vector comprising isolated nucleic acid for one or more gRNAs comprising a targeting sequence for a genomic target and nucleic acid comprising a coding sequence for a prophylactic or therapeutic gene product flanked by homology arms, or an effective amount of iii) isolated nucleic encoding Cas and nucleic acid for one or more gRNAs comprising a targeting sequence for a genomic target, e.g., a vector comprising isolated nucleic encoding Cas and nucleic acid for one or more gRNAs comprising a targeting sequence for a genomic target, and iv) isolated nucleic acid comprising a coding sequence for a prophylactic or therapeutic gene product flanked by homology arms, e.g., a vector comprising isolated nucleic acid comprising a coding sequence for a prophylactic or therapeutic gene product flanked by homology arms, wherein the expression of the coding sequence in the mammal prevents, inhibits or treats the disease or in the mammalian cell results in increased expression of the prophylactic or therapeutic gene product. In one embodiment, a composition comprises Cas9 or an isolated nucleic encoding Cas9, and isolated nucleic acid for one or more gRNAs comprising a targeting sequence for a genomic target and nucleic acid comprising a coding sequence for a prophylactic or therapeutic gene product flanked by homology arm. In one embodiment, a composition comprises isolated nucleic encoding Cas9 and nucleic acid for one or more gRNAs comprising a targeting sequence for a genomic target, and isolated nucleic acid comprising a coding sequence for a prophylactic or therapeutic gene product flanked by homology arms. In one embodiment, a Cas9 or an isolated nucleic encoding Cas9 and isolated nucleic acid for one or more gRNAs comprising a targeting sequence for a genomic target and nucleic acid comprising a coding sequence for a prophylactic or therapeutic gene product flanked by homology arm are separately administered, e.g., sequentially or at different locations. In one embodiment, isolated nucleic encoding Cas9 and nucleic acid for one or more gRNAs comprising a targeting sequence for a genomic target and isolated nucleic acid comprising a coding sequence for a prophylactic or therapeutic gene product flanked by homology arms are separately administered, e.g., sequentially or at different locations. In one embodiment, a Cas9 or an isolated nucleic encoding Cas9 and isolated nucleic acid for one or more gRNAs comprising a targeting sequence for a genomic target and nucleic acid comprising a coding sequence for a prophylactic or therapeutic gene product flanked by homology arm are administered at the same time and at the same location. In one embodiment, isolated nucleic encoding Cas9 and nucleic acid for one or more gRNAs comprising a targeting sequence for a genomic target and isolated nucleic acid comprising a coding sequence for a prophylactic or therapeutic gene product flanked by homology arms are administered at the same time and at the same location. In one embodiment, the disease is mucopolysaccharidosis, a lysosomal storage disease, hemophilia, thalassemia, or sickle cell disease. In one embodiment, the targeting sequence or homology arms are targeted to an intron. In one embodiment, one or more adeno-associated virus (AAV), adenovirus or lentivirus is/are employed to deliver at least one of Cas9 or an isolated nucleic encoding Cas9, or isolated nucleic acid for one or more gRNAs comprising a targeting sequence for a genomic target and nucleic acid comprising a coding sequence for a prophylactic or therapeutic gene product flanked by homology arms, or at least one of isolated nucleic encoding Cas9 and nucleic acid for one or more gRNAs comprising a targeting sequence for a genomic target, or isolated nucleic acid comprising a coding sequence for a prophylactic or therapeutic gene product flanked by homology arms. In one embodiment, a first rAAV delivers nucleic acid encoding Cas9. In one embodiment, a second rAAV delivers the nucleic acid comprising the targeting sequence and the coding sequence. In one embodiment, the first or second AAV is one of serotypes AAV1-9 or AAVrh10. In one embodiment, the first and the second rAAVs are different serotypes. In one embodiment, the mammal is a human. In one embodiment, one or more of the gRNAs target the albumin locus, the Rosa26 locus, AAVS1 locus, CCR5 locus, HPRT locus, or alpha fetoprotein locus. In one embodiment, the disease is mucopolysaccharoidosis type I, type II type III, type IV, type V, type VI or type VII. In one embodiment, the disease is Tay-Sachs disease or Sandhoff disease (GM2-gangliosidosis disease). In one embodiment, the coding sequence encodes iduronidase, beta-globin, iduronate, beta galactosidase, sulfatase, hexM, hexoaminidase A or hexosaminidase B. In one embodiment, the intron is an albumin gene intron. In one embodiment, the intron is the first intron. In one embodiment, the targeting sequence is promoterless, e.g., until inserted into the host cell genome. In one embodiment, the targeting sequence targets sequences within the first 500, 400, 300, 200, or 100 nucleotides of the intron. In one embodiment, the Cas9 comprises Streptococcus pyogenes (SpCas9), Staphylococcus aureus (SaCas9), Streptococcus thermophilus (StCas9), Neisseria meningitidis (NmCas9), Francisella novicida (FnCas9),Campylobacter jejuni (CjCas9), CasX and CasY, Cas12a (Cpf1), Cas14a, eSpCas9, SpCas9-HF1, HypaCas9, Fokl-Fused dCas9, or xCas9. In one embodiment, liposomes are employed to deliver Cas9 or an isolated nucleic encoding Cas9, isolated nucleic acid for one or more gRNAs comprising a targeting sequence for a genomic target and nucleic acid comprising a coding sequence for a prophylactic or therapeutic gene product flanked by homology arms, isolated nucleic encoding Cas9 and nucleic acid for one or more gRNAs comprising a targeting sequence for a genomic target, isolated nucleic acid comprising a coding sequence for a prophylactic or therapeutic gene product flanked by homology arms, or any combination thereof. In one embodiment, the nucleic acid comprising a coding sequence for a prophylactic or therapeutic gene product is not operably linked to a promoter. In one embodiment, at least one of Cas9 or an isolated nucleic encoding Cas9, isolated nucleic acid for one or more gRNAs comprising a targeting sequence for a genomic target and nucleic acid comprising a coding sequence for a prophylactic or therapeutic gene product flanked by homology arms, isolated nucleic encoding Cas9 and nucleic acid for one or more gRNAs comprising a targeting sequence for a genomic target, or isolated nucleic acid comprising a coding sequence for a prophylactic or therapeutic gene product flanked by homology arms is delivered parenterally. In one embodiment, at least one of Cas9 or an isolated nucleic encoding Cas9, isolated nucleic acid for one or more gRNAs comprising a targeting sequence for a genomic target and nucleic acid comprising a coding sequence for a prophylactic or therapeutic gene product flanked by homology arms, isolated nucleic encoding Cas9 and nucleic acid for one or more gRNAs comprising a targeting sequence for a genomic target, or isolated nucleic acid comprising a coding sequence for a prophylactic or therapeutic gene product flanked by homology arm is delivered intravenously. For example, Cas protein may be delivered via a different route that one of the isolated nucleic acids. In one embodiment, a single administration is effective to prevent, inhibit or treat a disease, or one or more symptoms thereof, in a mammal. In one embodiment, a dose of virus may be from about 1 x 10¹² vg/kg to about 1 x 10¹⁴ vg/kg, e.g., about 3 x 10¹² vg/kg to about 5x10¹³ vg/kg. In one embodiment, the ratio of Cas vector to the donor vector is about 1:20, 1:15, 1:10, 1:8, 1:6, 1:5, 1: 2 or 1:1. in one embodiment, the ratio of Cas encoding viral particles to donor nucleic acid containing viral particles is about 1:20, 1:15, 1:10, 1:8, 1:6, 1:5, 1: 2 or 1:1.

Further provided is a composition comprising a first rAAV comprising an isolated nucleic encoding Cas, e.g., Cas9, and a second rAAV comprising an isolated nucleic comprising sequences for one or more gRNAs comprising a selected targeting sequence and a selected coding sequence flanked by homology arms, or a first rAAV comprising an isolated nucleic encoding Cas, e.g., Cas9, and an isolated nucleic comprising sequences for one or more gRNAs comprising a selected targeting sequence and a second rAAV comprising a selected coding sequence flanked by homology arms.

In one embodiment, one or more CRISPR components and the gene of interest are delivered using viral vectors, e.g., one or more lentivirus vectors or two rAAV vectors. In one embodiment, the rAAV vector is a rAAV2, rAAV5, rAAV6, rAAV8, or rAAV9 vector. In one embodiment, the rAAVs are administered to an embryo, a fetus, an infant (e.g., a human that is 3 years old or less such as less than 3, 2.5, 2, or 1.5 years of age), a pre-adolescent (e.g., in humans those less than 10, 9, 8, 7, 6, 5, or 4 but greater than 3 years of age), or adult (e.g., humans older than about 12 years of age).

In one embodiment, the mammal is a human. In one embodiment, multiple doses are administered. In one embodiment, the composition is administered weekly, monthly or two or more months apart. In one embodiment, a single dose is administered.

In one embodiment, the amount of vector(s) administered results in an increase, e.g., at least 2-, 5-, 10-, 25-, 50-, 100-, 200- or 500-fold or more, up to 1000-fold of the gene product, e.g., in plasma or tissue, e.g., the brain, in the mammal relative to a corresponding mammal with that is not administered the vectors.

Diseases that may be prevented, inhibited or treated using the methods disclosed herein include, but are not limited to, Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich’s ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy (a deficiency of survivor of motor neuron -1, SMN-1), Spinocerebellar ataxia, Tangier disease, Tay-Sachs disease, Tuberous sclerosis, Von Hippel-Lindau syndrome, Williams syndrome, Wilson’s disease, or Zellweger syndrome. In one embodiment, the disease is a lysosomal storage disease, e.g., a lack or deficiency in a lysosomal storage enzyme. Lysosomal storage diseases include, but are not limited to, mucopolysaccharidosis (MPS) diseases, for instance, mucopolysaccharidosis type I, e.g., Hurler syndrome and the variants Scheie syndrome and Hurler-Scheie syndrome (a deficiency in alpha-L-iduronidase); Hunter syndrome (a deficiency of iduronate-2-sulfatase); mucopolysaccharidosis type III, e.g., Sanfilippo syndrome (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV, e.g., Morquio syndrome (a deficiency of galactosamine-6-sulfate sulfatase or beta-galactosidase); mucopolysaccharidosis type VI, e.g., Maroteaux-Lamy syndrome (a deficiency of arylsulfatase B); mucopolysaccharidosis type II; mucopolysaccharidosis type III (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV (A or B; a deficiency of galactosamine-6-sulfatase and beta-galatacosidase); mucopolysaccharidosis type VI (a deficiency of arylsulfatase B); mucopolysaccharidosis type VII (a deficiency in beta-glucuronidase); mucopolysaccharidosis type VIII (a deficiency of glucosamine-6-sulfate sulfatase); mucopolysaccharidosis type IX (a deficiency of hyaluronidase); Tay-Sachs disease (a deficiency in alpha subunit of beta-hexosaminidase); Sandhoff disease (a deficiency in both alpha and beta subunit of beta-hexosaminidase); GM1gangliosidosis (type I or type II); Fabry disease (a deficiency in alpha galactosidase); metachromatic leukodystrophy (a deficiency of aryl sulfatase A), Pompe disease (a deficiency of acid maltase); fucosidosis (a deficiency of fucosidase); alpha-mannosidosis (a deficiency of alpha-mannosidase); beta-mannosidosis (a deficiency of beta-mannosidase), neuronal ceroid lipofuscinosis (NCL) (a deficiency of ceroid lipofucinoses (CLNs), e.g., Batten disease having a deficiency in the gene product of one or more of CLN1 to CLN14), and Gaucher disease (types I, II and III; a deficiency in glucocerebrosidase), as well as disorders such as Hermansky-Pudiak syndrome; Amaurotic idiocy; Tangier disease; aspartylglucosaminuria; congenital disorder of glycosylation, type la; Chediak-Higashi syndrome; macular dystrophy, corneal, 1; cystinosis, nephropathic; Fanconi-Bickel syndrome; Farber iipogranuiomatosis; fibromatosis; geleophysic dysplasia; glycogen storage disease I; glycogen storage disease lb; glycogen storage disease Ic; glycogen storage disease III; glycogen storage disease IV; glycogen storage disease V; glycogen storage disease VI; glycogen storage disease VII; glycogen storage disease 0; immunoosseous dysplasia, Schimke type; lipidosis; lipase b; mucolipidosis II; mucolipidosis II, including the variant form; mucolipidosis IV; neuraminidase deficiency with beta-galactosidase deficiency; mucolipidosis I; Niemann-Pick disease (a deficiency of sphingomyelinase); Niemann-Pick disease without sphingomyelinase deficiency (a deficiency of a npc1 gene encoding a cholesterol metabolizing enzyme); Refsum disease; Sea-blue histiocyte disease; infantile sialic acid storage disorder; sialuria; multiple sulfatase deficiency; triglyceride storage disease with impaired longchain fatty acid oxidation; Winchester disease; Wolman disease (a deficiency of cholesterol ester hydrolase); Deoxyribonuclease l-like 1 disorder; arylsulfatase E disorder; ATPase, H+ transporting, lysosomal, subunit 1disorder; glycogen storage disease IIb; Ras-associated protein rab9 disorder; chondrodysplasia punctata 1, X-linked recessive disorder; glycogen storage disease VIII; lysosome-associated membrane protein 2 disorder; Menkes syndrome; congenital disorder of glycosylation, type Ic; and sialuria. Replacement of less than 20%, e.g., less than 10% or about 1% to 5% levels of lysosomal storage enzyme found in nondiseased mammals, may prevent, inhibit or treat neurological symptoms such as neurological degeneration in mammals. In one embodiment, the disease to be prevented, inhibited or treated with a particular gene includes, but is not limited to, MPS I (IDUA), MPS II (IDS), MPS IIIA (Heparan-N-sulfatase;sulfaminidase), MPS IIIB (alpha-N-acetyl-glucosaminidase), MPS IIIC (Acetyl-CoA:alpha -N-acetyl-glucosaminide acetyltransferase), MPS IIID (N-acetylglucosamine 6-sulfatase), MPS VII (beta-glucoronidase), Gaucher (acid beta-glucosidase), Alpha-mannosidosis (alpha-mannosidase), Beta-mannosidosis (beta-mannosidase), Alpha-fucosidosis (alpha-fucosidase), Sialidosis (alpha-sialidase) , Galactosialidosis (Cathepsin A), Aspartylglucosaminuria (aspartylglucosaminidase), GM1-gangliosidosis (beta-galactosidase), Tay-Sachs (beta-hexosaminidase subunit alpha), Sandhoff (beta-hexosaminidase subunit beta), GM2-gangliosidosis/variant AB (GM2 activator protein), Krabbe (galactocerebrosidase), Metachromatic leukodystrophy (arylsulfatase A) ,hemophilia (factor VIII or factor IX), thalassemia (HBB, HBA1, or HBA2), sickle cell anemia (HBB), von Willenbrand disease (von Willenbrand factor), and other disorders including but not limited to Alzheimer’s disease (expression of an antibody, such as an antibody to beta-amyloid, or an enzyme that attacks the plaques and fibrils associated with Alzheimer’s), or Alzheimer’s and Parkinson’s diseases (expression of neuroprotective proteins including but not limited to GDNF or Neurturin). In one embodiment, the gene encodes factor VIII. In one embodiment, the gene encodes factor IX. In one embodiment, the gene encodes beta-globin. In one embodiment, the gene encodes alpha-globin.

### BRIEF DESCRIPTION OF THE FIGURES

FIGS. 1A-B. Construct design and validation in MPS I mice through hydrodynamic injection. (A) Sequence of AAV vectors represented in cartoon.hAAT: human α1-antitrypsin promoter; ITR: inverted terminal repeats; SA: splice acceptor; SD; splice donor; PA: poly A; HA: homology arm; IDUA: human IDUA cDNA; RE: restriction enzyme site; U6: U6 promoter sequence. (B) The two plasmids were administered into MPS. I mice through hydrodynamic injection. Two days post injection, the treated mice and controls were euthanized for IDUA enzyme assay. The enzyme activities in mice receiving Cas9 and donor plasmids were significantly higher than those in untreated or mice receiving the donor plasmid only.

FIG. 2. Ganglioside accumulation in the cortex of MPS 1 mouse brains. MP$ I mice had a significant accumulation of GM2(18:0) & GM2(20:0) and GM3(20:0). Output was processed and reported as the peak area ratios of the analytes to the corresponding internal standard. Data are mean ± standard errors.

FIG. 3. PCR with these two set of primers to confirm integration. Two sets of primers were designed to detect insertion of donor sequence through HDR or NHEJ mechanism. FP1&2:forward primer 1&2; RP1&2: reverse primer 1&2. The amplicons are sequenced for further confirmation.

FIGS. 4A-B. Metabolomics and proteomics profiling of mice with lysosomal diseases. (A) Principle component analysis of global metabolomics profiles of SD and normal mice with RPLC. The metabolites identified in the brain of mice (n=3 for each group) were analyzed through the Matlab software. (B) Proteomics profiling of MPS I mouse whole brain. The spots that were significantly different in the 2D gel were isolated for LC-MS/MS, resulting in identification of 47 dysregulated proteins.

FIG. 5. Map of insertion site in albumin locus (SEQ ID NO:1).

FIG. 6. Neonatal injection of AAV vectors carrying the CRISPR system into MPS I mice achieved 1920-fold of wildtype activities.

FIG. 7. Exemplary vectors. hAAT: human α1-antitrypsin promoter; TBG: thyroxine-binding globulin; ITR: inverted terminal repeats; SA: splice acceptor; SD; splice donor; PA: polyA; HA: homology arm; RE: restriction enzyme site; U6: U6 promoter sequence.

FIG. 8. Exemplary vectors and promoters.

FIG. 9. Exemplary vector for MPSI study.

FIG. 10. Doses for MPSI mice.

FIG. 11. The system achieves 1.5 fold of plasma IDUA level with 4.7% of positive control (e.g., the use of 3 vectors one of which encodes a nuclease).

FIG. 12. Tissue IDUA levels increased at 1 month post-dosing.

FIG. 13. Tissue GAG levels normalized at 1 month post-dosing.

FIG. 14. Genome editing events detected at the target locus at 1 month post-dosing.

FIG. 15. Fear conditioning showed that treated MPS I mice had better memory and learning ability. Baseline is generalized fear in an altered context in the absence of the cues. Cued freezing is measured in an altered context and is the freezing specific to the paired cues. The difference of context and cue from baseline determines how robust the memory is.

FIG. 16. Pole test showed that treated MPS I mice had better neuromotor function.

FIG. 17. Kaplan Meier curve showed that the survival rate of treated MPS I mice was better.

FIG. 18. Vector for Sandhoff testing.

FIG. 19. Sandhoff and Tay-Sachs diseases. HexA is a heterodimer (alpha and beta subunits). HexM (a beta-alpha hybrid) is a homodimer.

FIG. 20. Plasma Hex enzyme activities after AAV injection of Cas9 + Donor (middle dose).

FIG. 21. Tissue Hex enzyme activities increased 4 month post dosing.

FIGS. 22A-D. Tissue GM2 gangliosides reduced 4 month post dosing.

FIG. 23. Rotarod analysis showed that treated SD mice had significant improved performance (better motor function and coordination). * means p<0.05 when comparing treated SD mice to untreated SD mice.

FIG. 24. Histological analysis showed that cellular vacuolation was reduced in the brain and liver of treated SD mice. The brain and liver were processed for H&E staining (upper and middle panel), and immunohistochemisty for Hex A enzyme (lower panel). Treated SD mice, untreated SD and normal mice are shown in the left, middle and right columns, respectively. Kupffer cell vacuolation (small, well defined, vesicles with clear to pale-eosinophilic content) in the liver of untreated SD mice was reduced in treated SD mice. In the cerebellum, pons, thalamus, hypothalamus and brain cortex of untreated SD mice, there was neuronal vacuolation, which was minimal to mild in treated SD and normal mice. When the brain was stained against Hex A proteins, the signal intensity in 1 out of 3 treated SD mice was comparable to normal mice, while only minimal signal was observed in untreated SD mice. Objective x40.

FIGS. 25A-C. Construct design and gRNA validation by Surveyor assay. (A) Sequence of AAV vectors represented in cartoon. TBG: thyroxine-binding globulin; ITR: inverted terminal repeats; SA: splicing acceptor; SD: splicing donor; PA: polyA; ITR: inverted terminal repeat; HA: homology arm; IDUA: human IDUA cDNA; RE: restriction enzyme site; U6: U6 promoter. (B) SURVEYOR assay for gRNA activity in MEF cells. (C) Hex total activity in the liver increased significantly 2 days after hydrodynamic injection of AAV-SaCas9 and AAV-HEXB-gRNA plasmids into SD mice (n=3). * means p<0.05 when comparing treated SD mice to untreated SD mice.

FIGS. 26A-D. Hydrodynamic injection of plasmids encoding HEXM sequence into adult SD mice. Hex A and total activities in the liver and brain of treated mice increased significantly 2 days post-dosing. * means p<0.05 when comparing treated SD mice to untreated SD mice.

FIGS. 27A-D. Plasma and tissue Hex enzyme activities increased significantly after AAV injection. Plasma Hex A (A) and total (B) activities significantly increased on Day 30, 60 and 90 post dosing. Four months post dosing, all mice were euthanized after transcardial perfusion. The brain, liver, heart and spleen were harvested for enzyme assays. Tissue Hex A (C) and total (D) activities increased significantly. * means p<0.05 when comparing treated SD mice to untreated SD mice.

FIGS. 28A-D. Tissue GM2 gangliosides reduced 4 months post dosing. GM2 gangliosides in the brain (A), heart (B), liver (C) and spleen (D) were quantified by HPLC-MS/MS. * means p<0.05 when comparing treated SD mice to untreated SD mice.w

FIG. 29. Schematic of positions of albumin gRNAs (SEQ ID NO:2).

FIG. 30. β-gal enzyme activity following hydrodynamic injection of plasmids encoding Cas9/gRNA and human GLB1 donor.

FIG. 31. β-galactosidase enzyme activity in plasma over 120 days.

FIG. 32. Tissue β-gal enzyme activity after 120 days - Middle dose only.

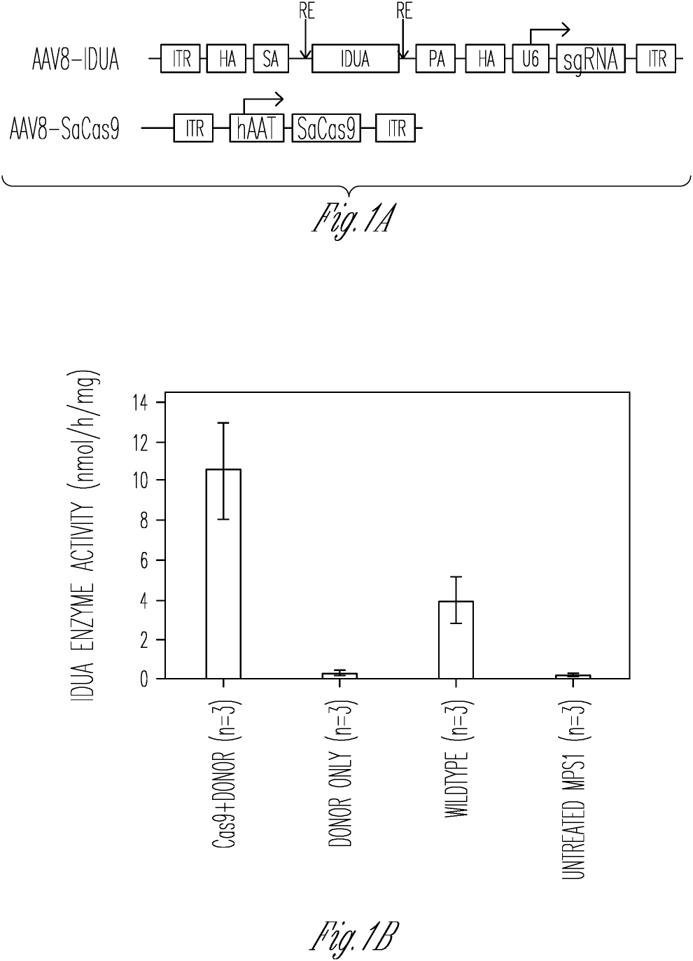

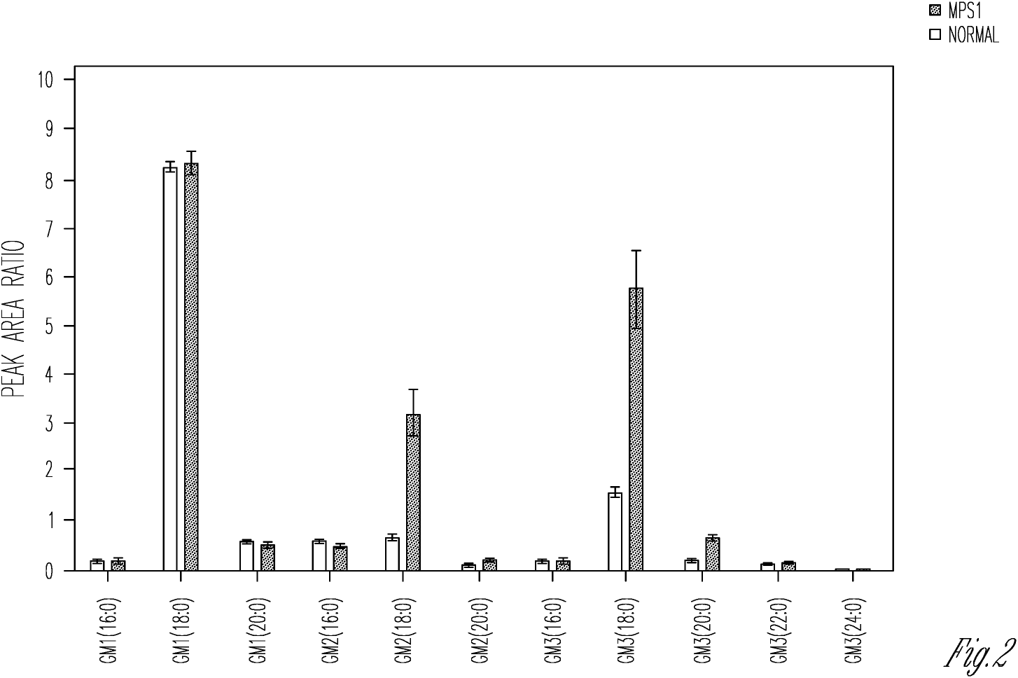

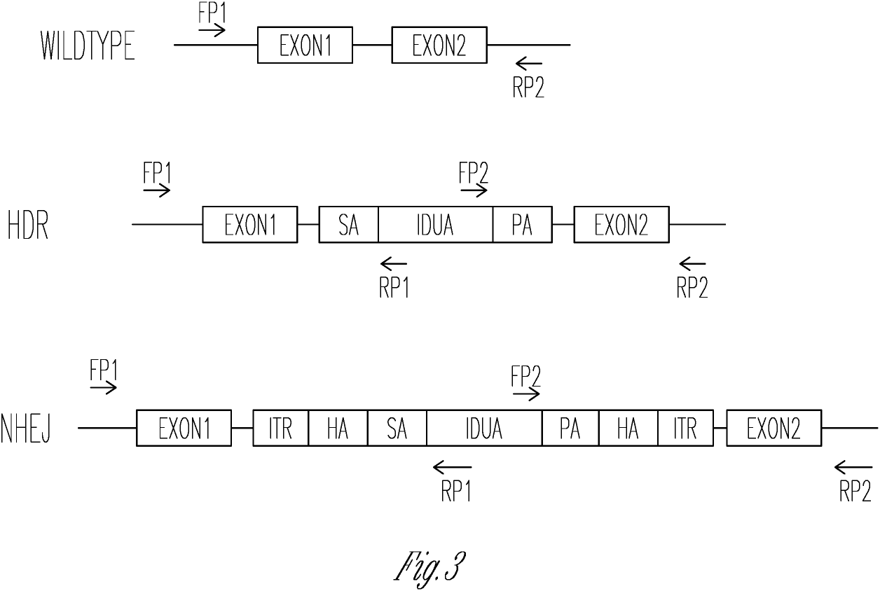

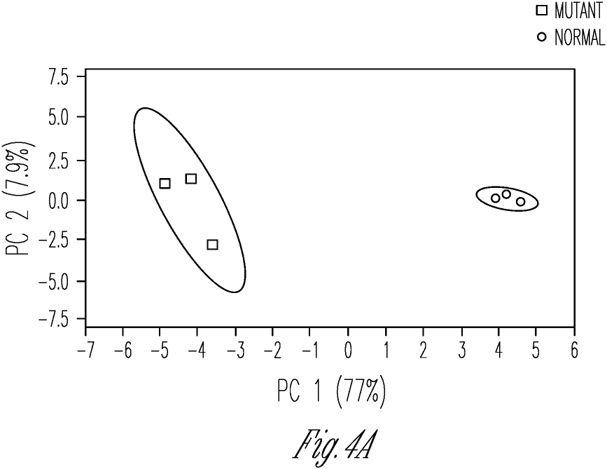

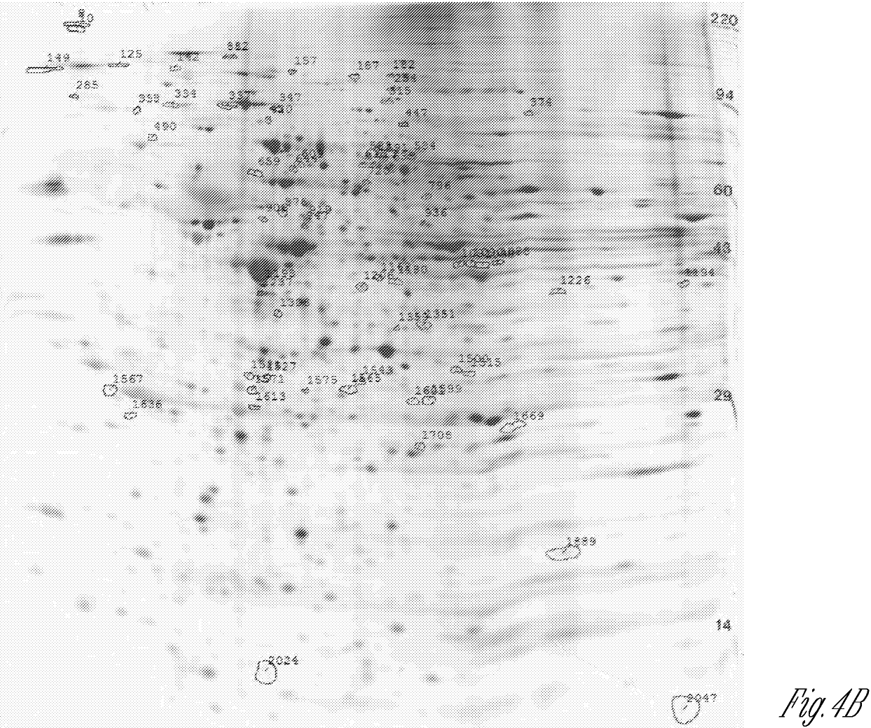

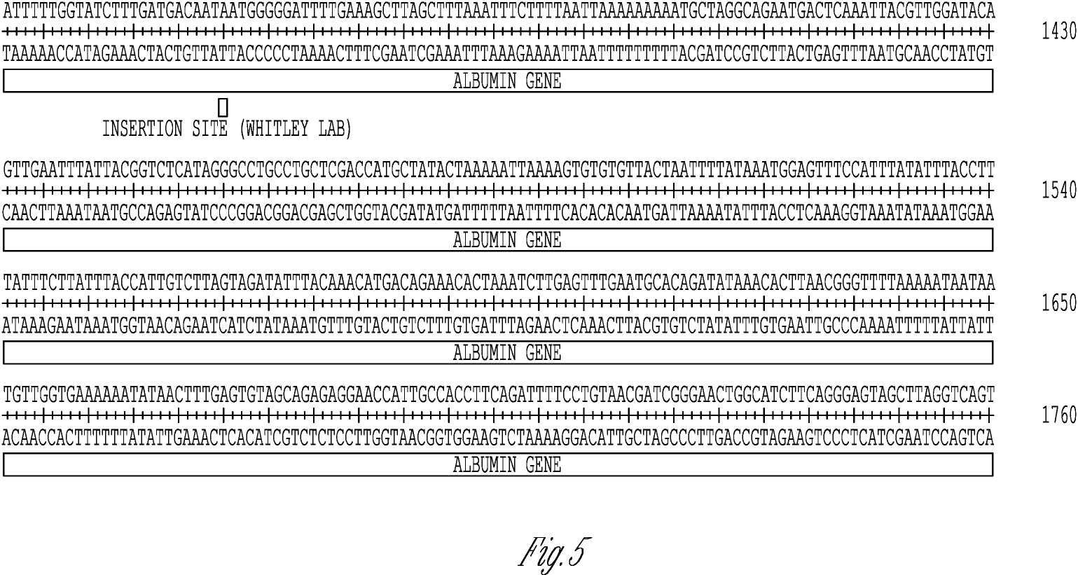

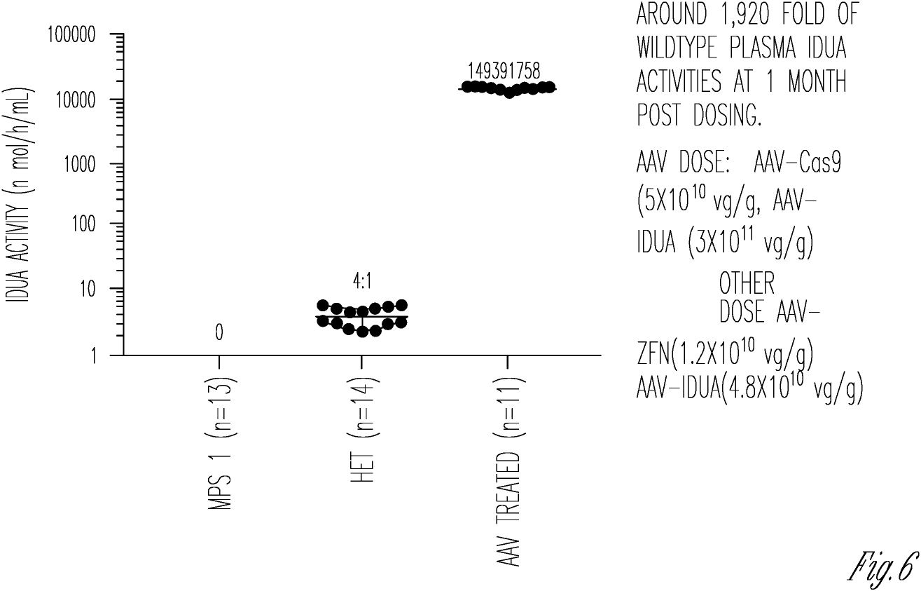

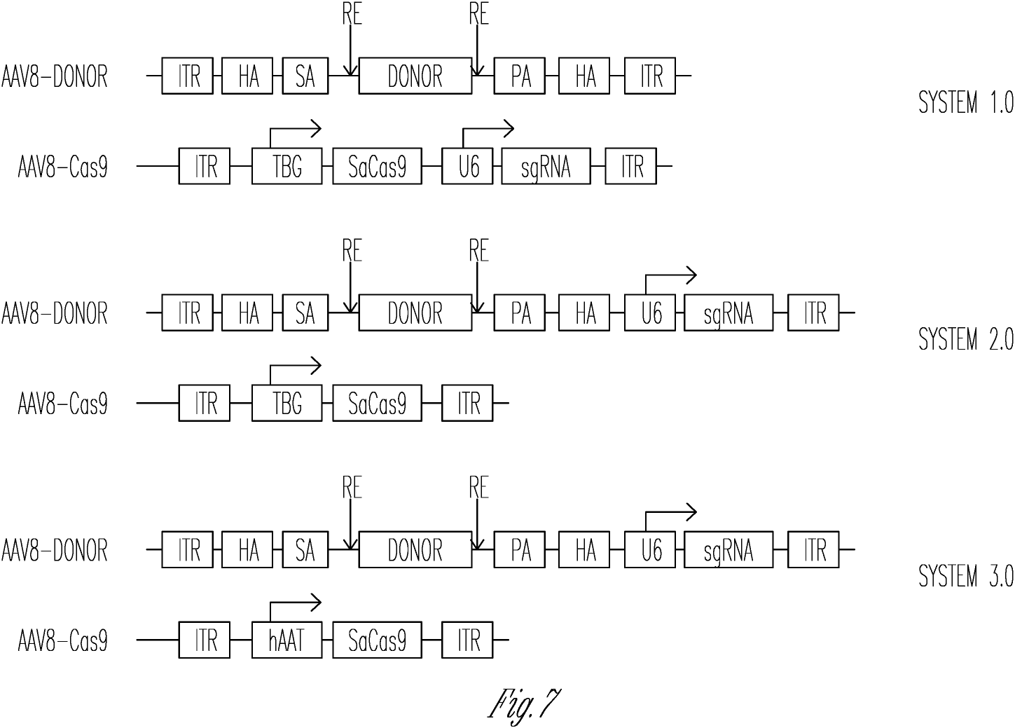

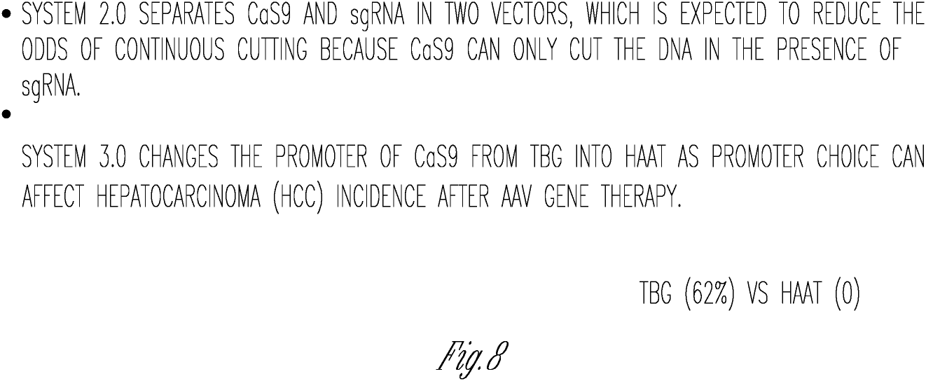

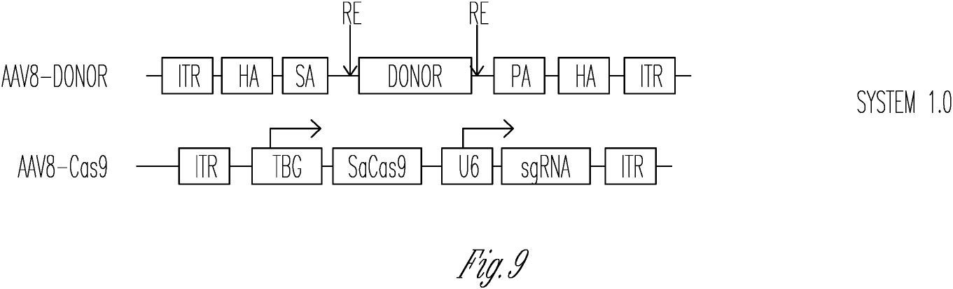

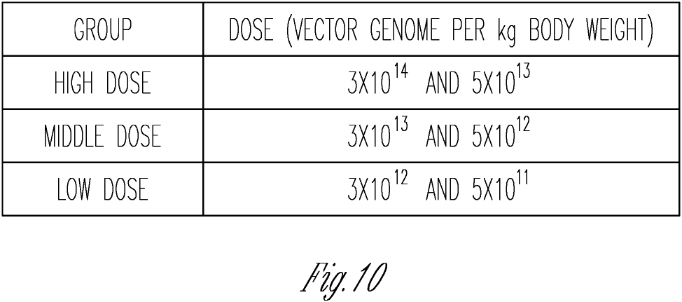

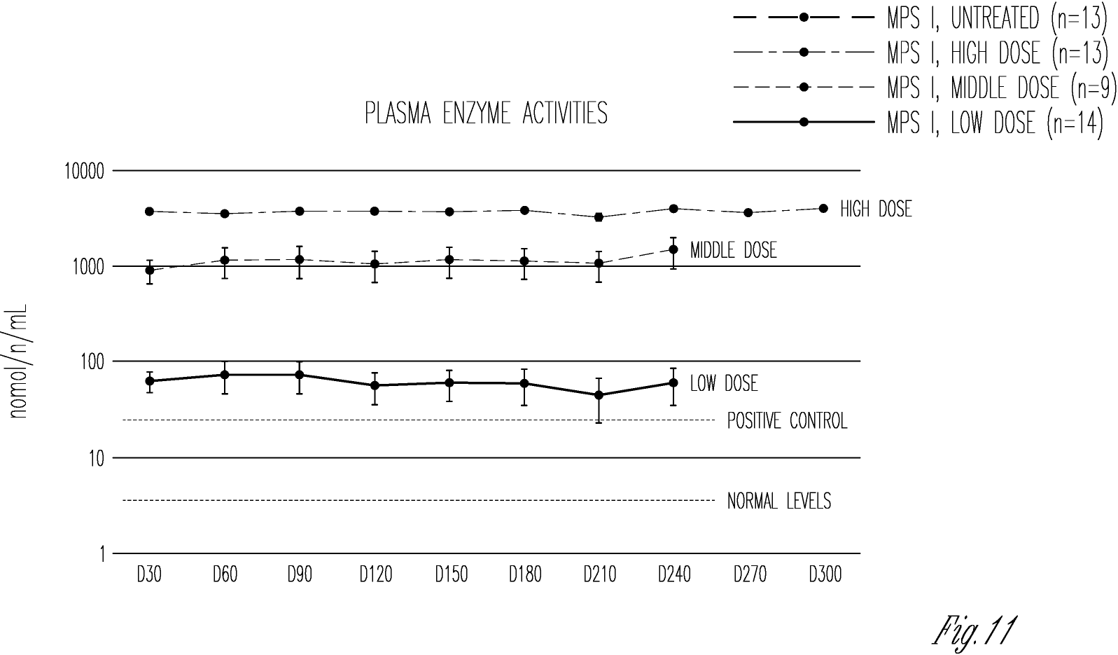

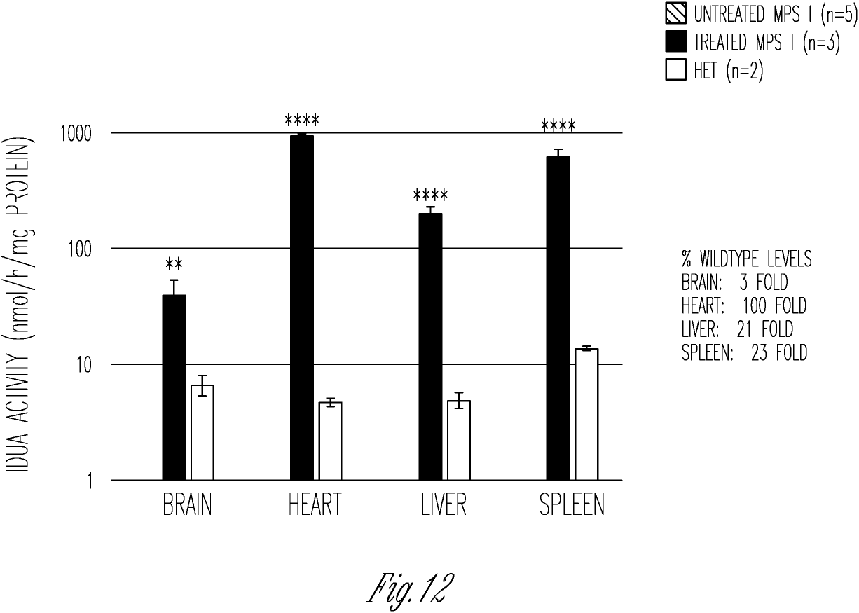

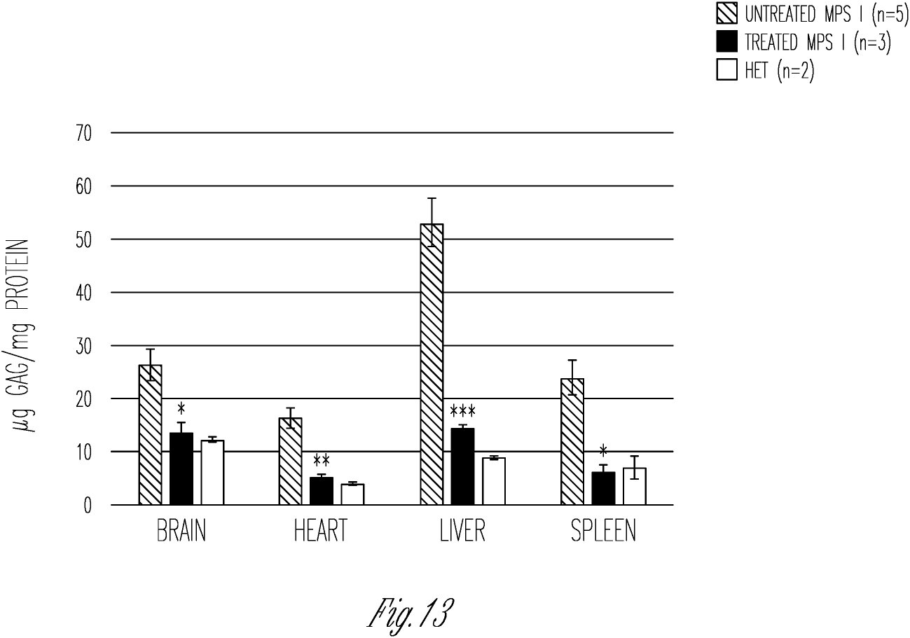

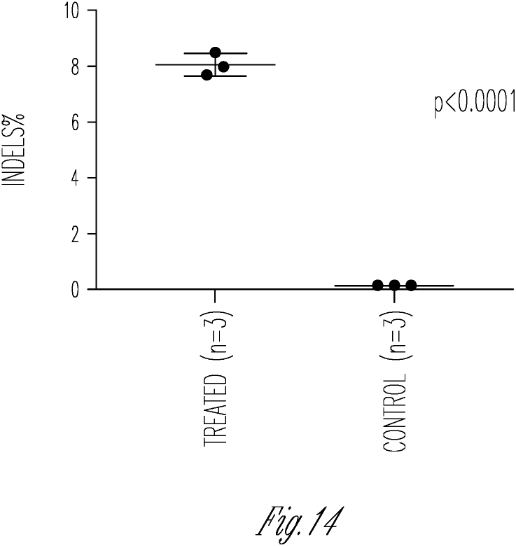

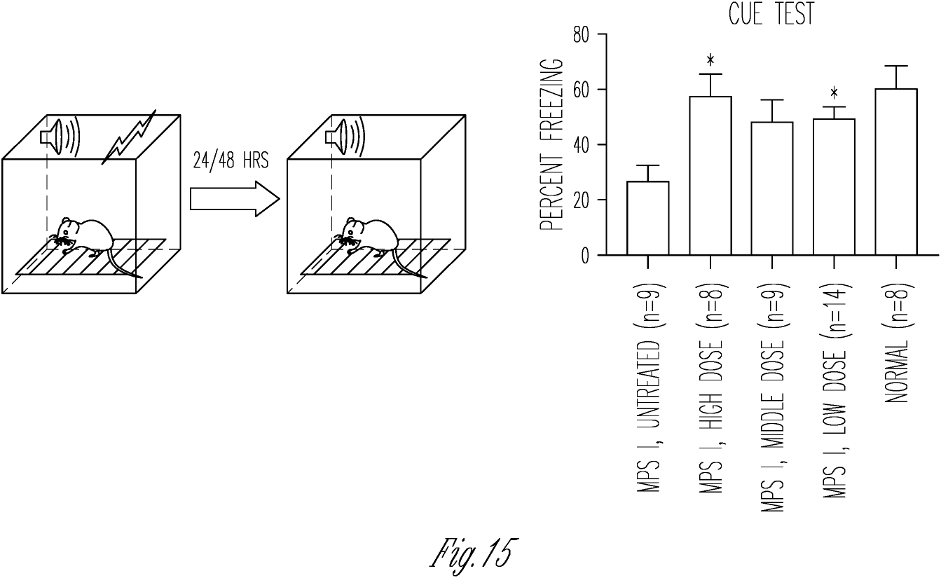

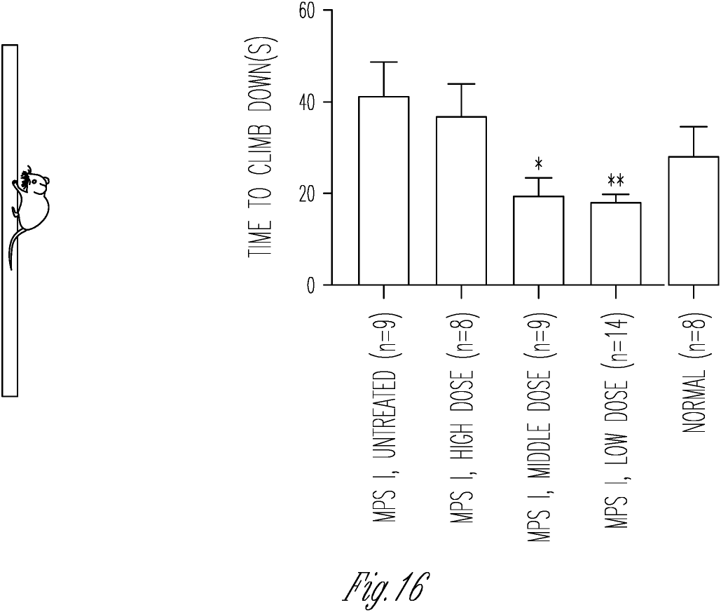

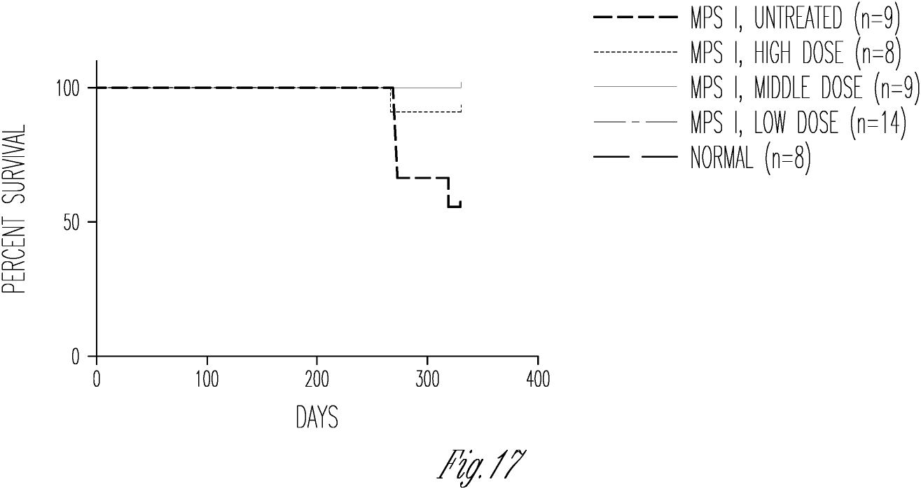

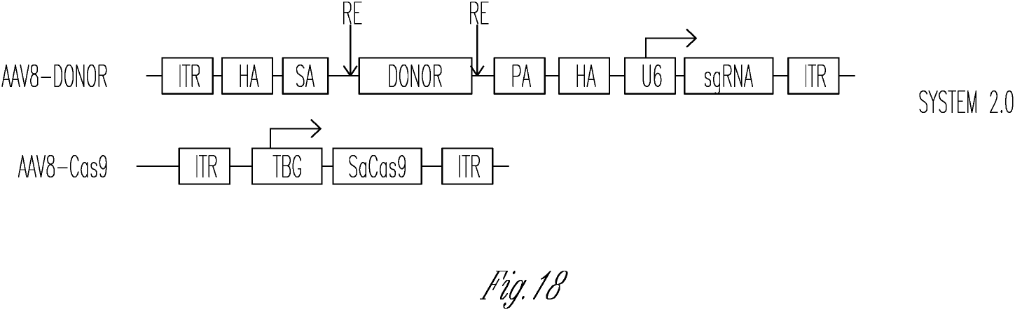

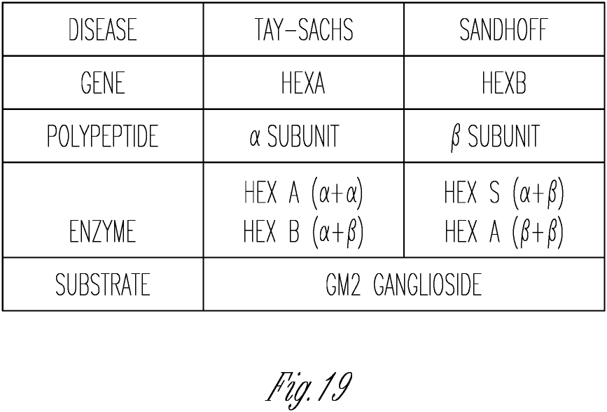

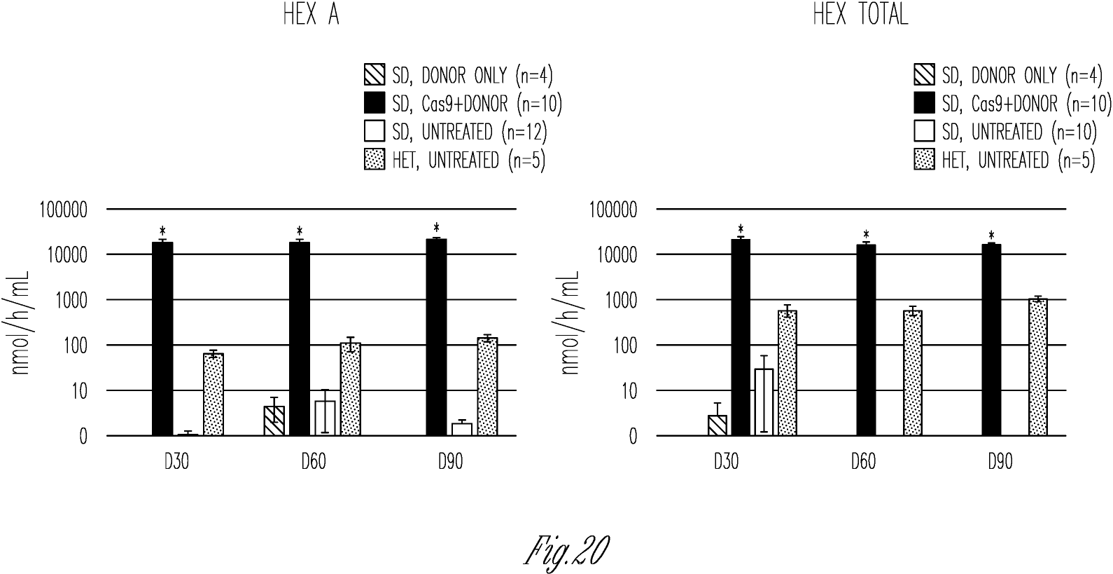

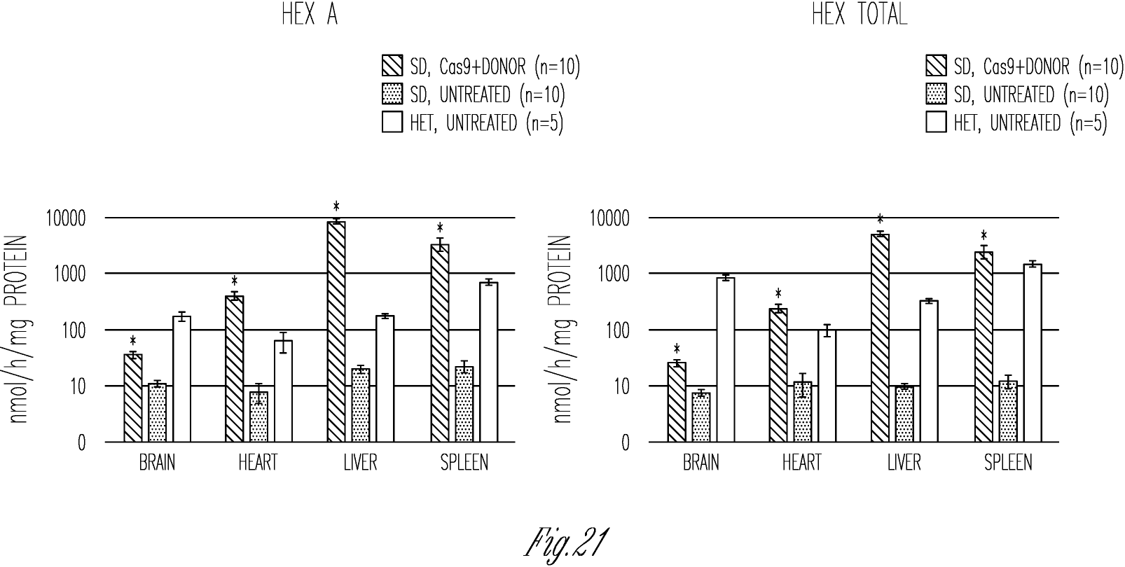

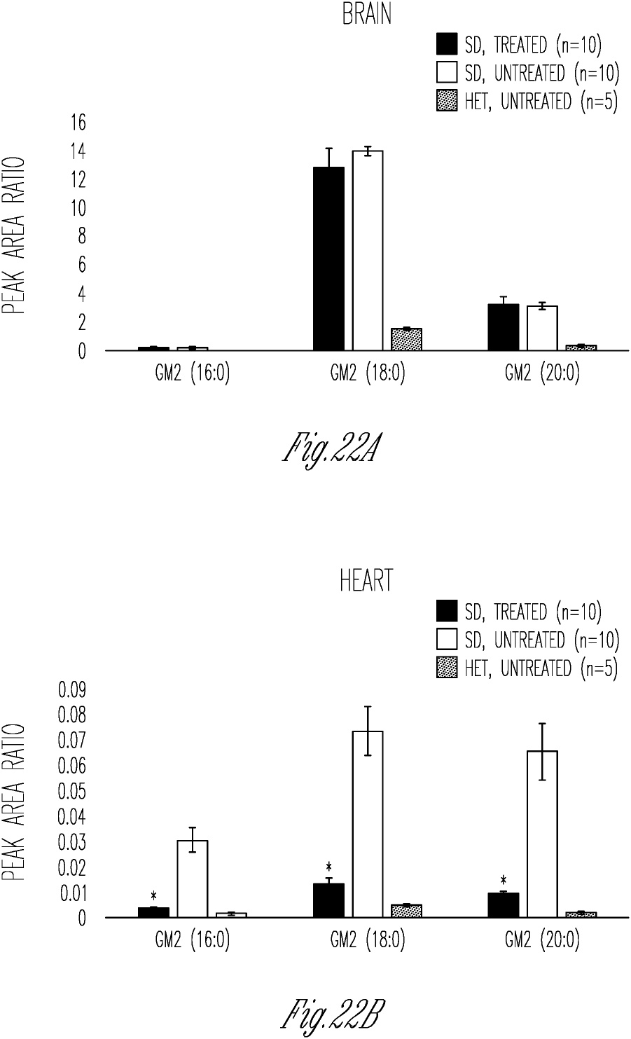

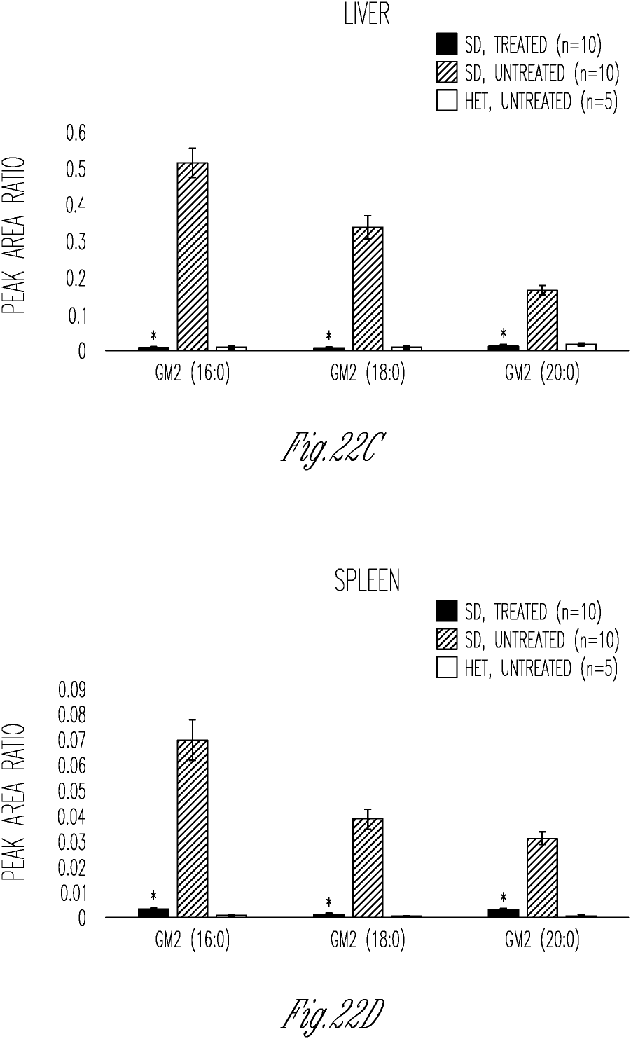

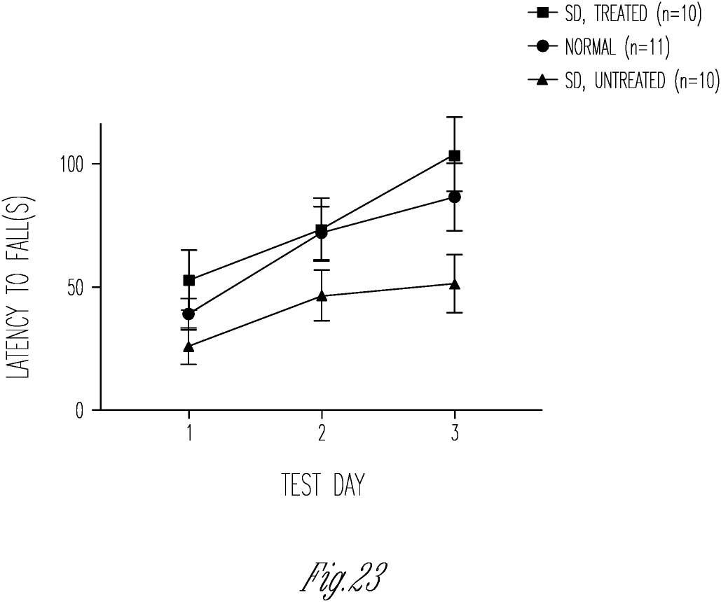

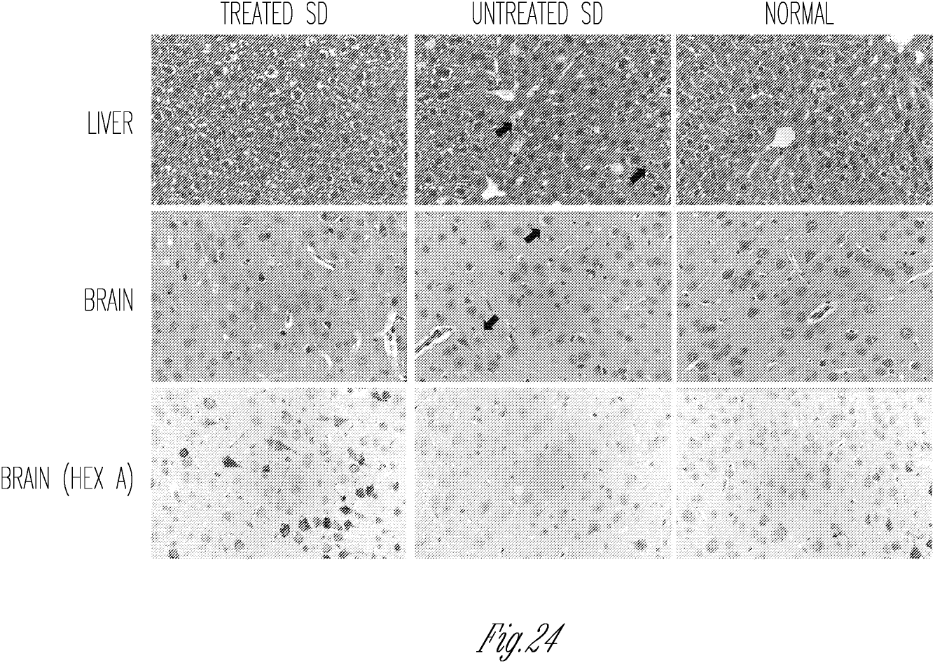

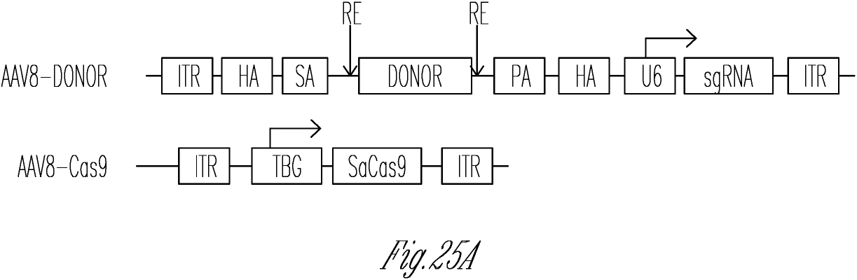

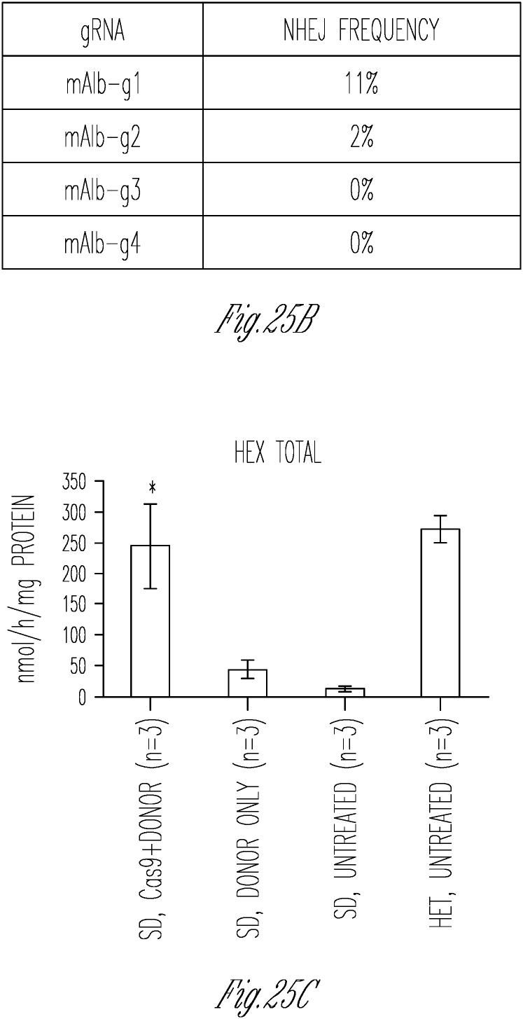

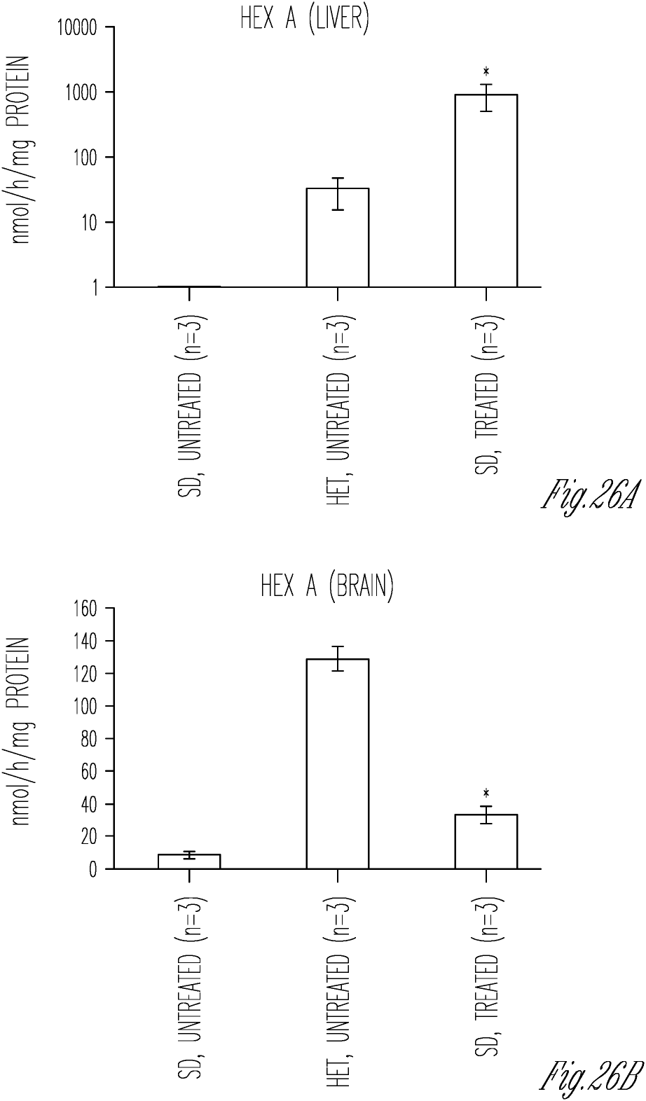

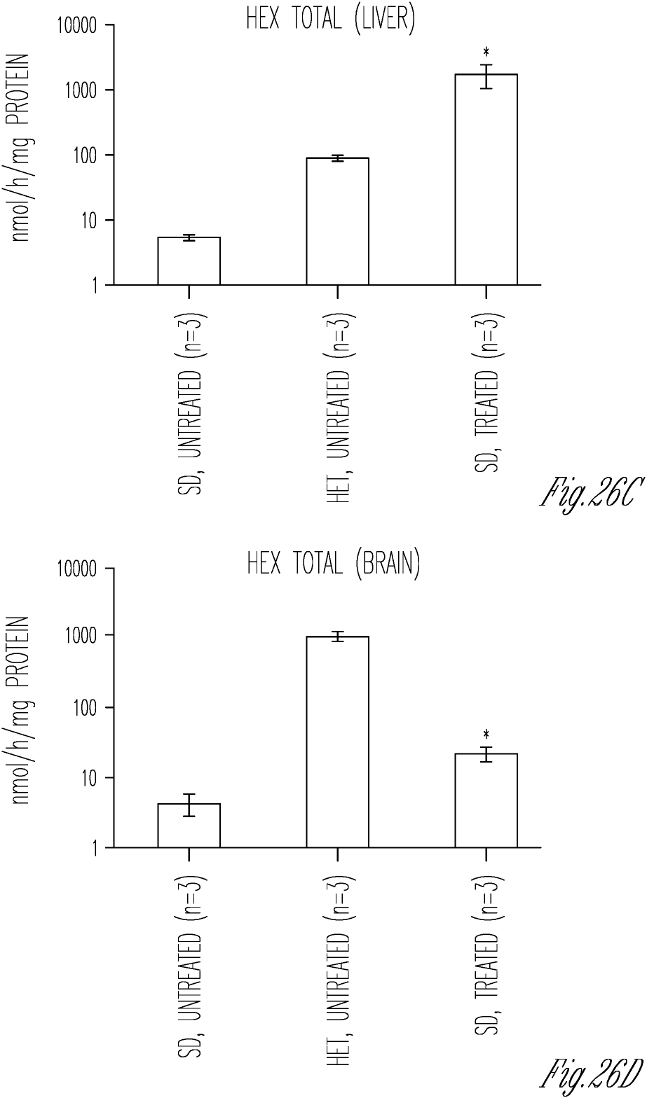

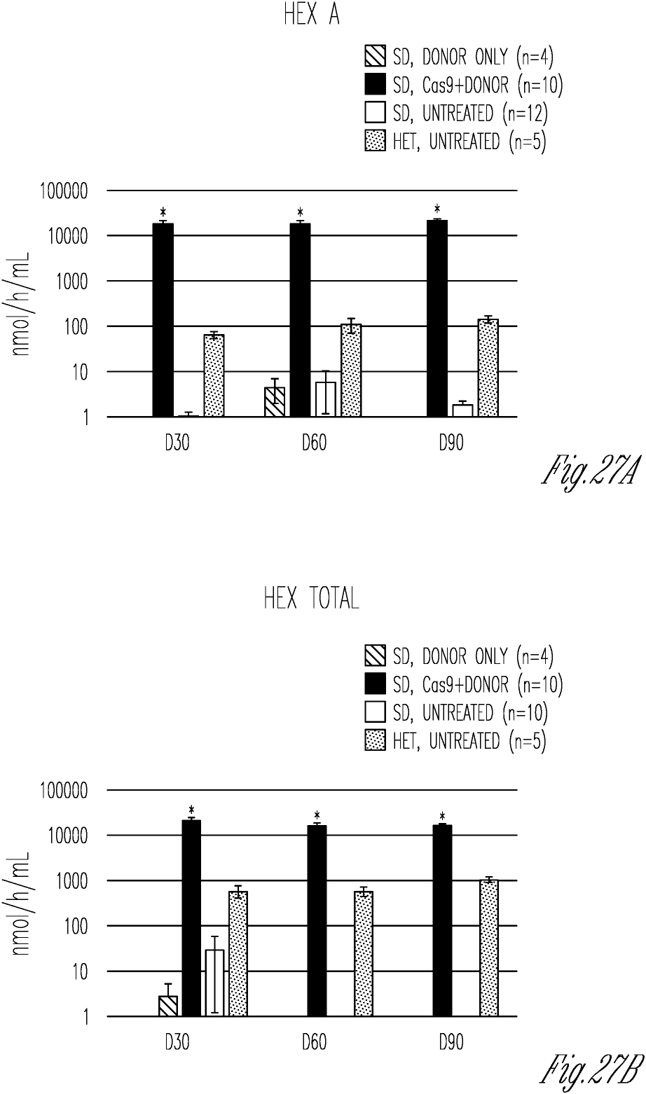

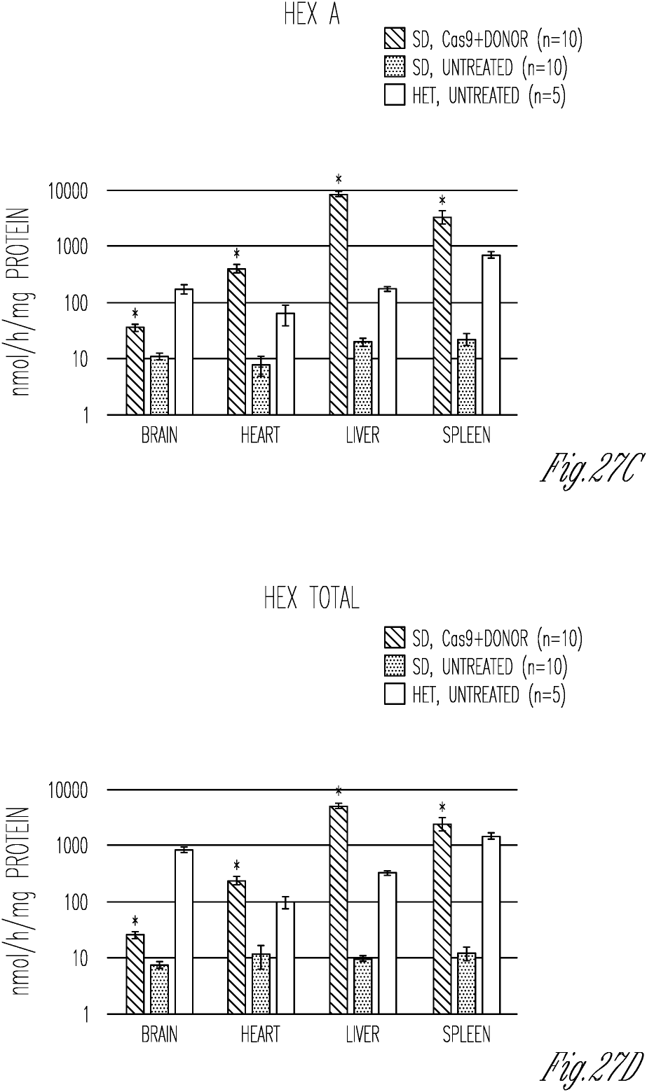

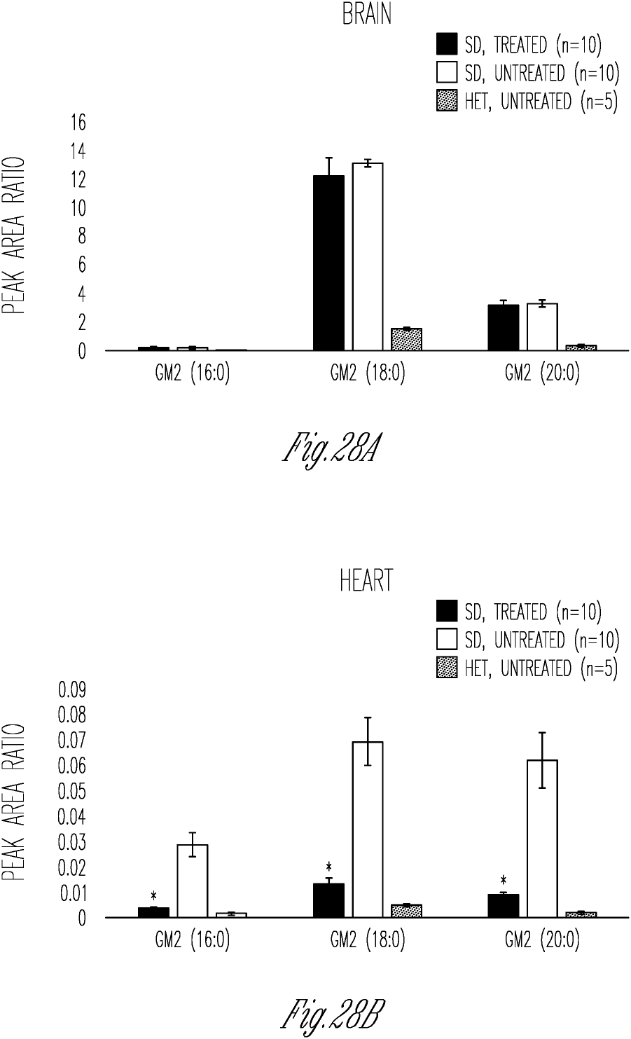

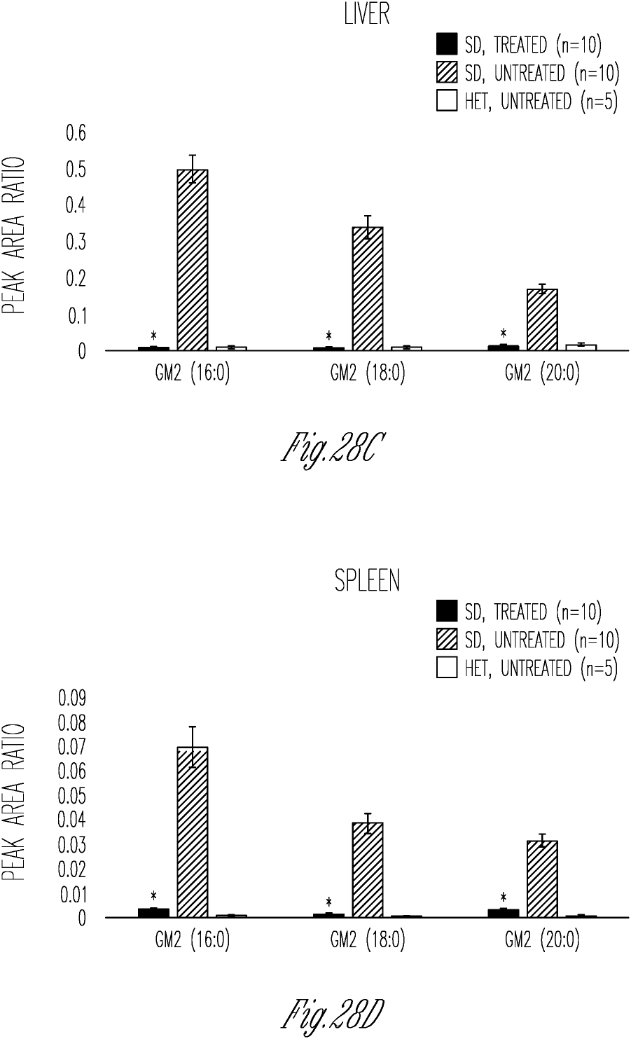

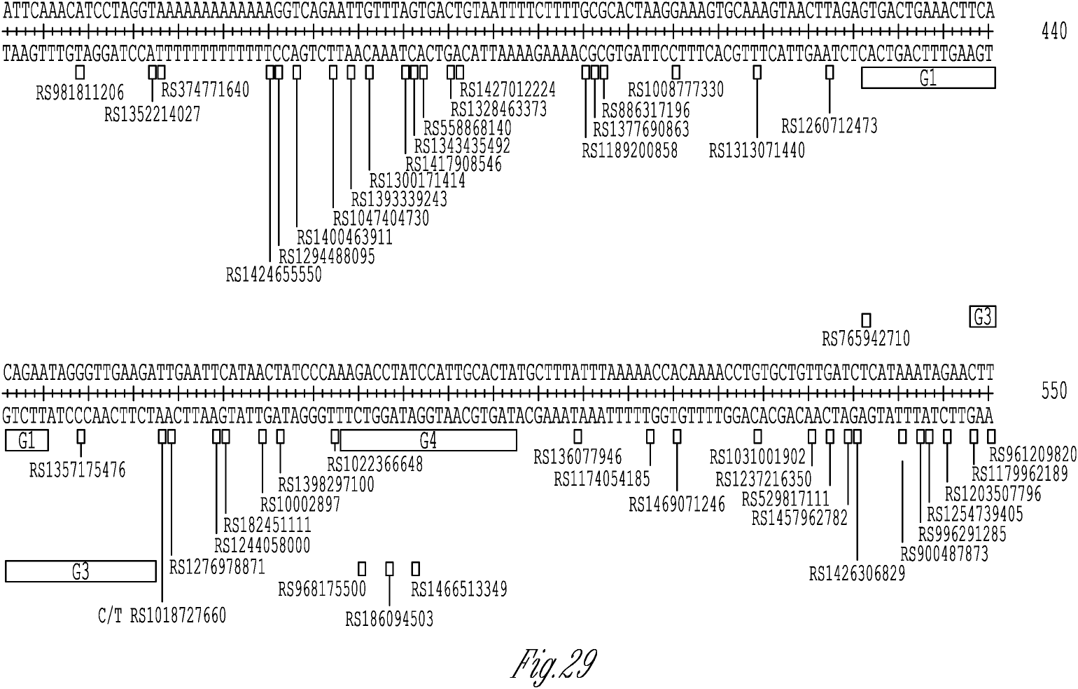

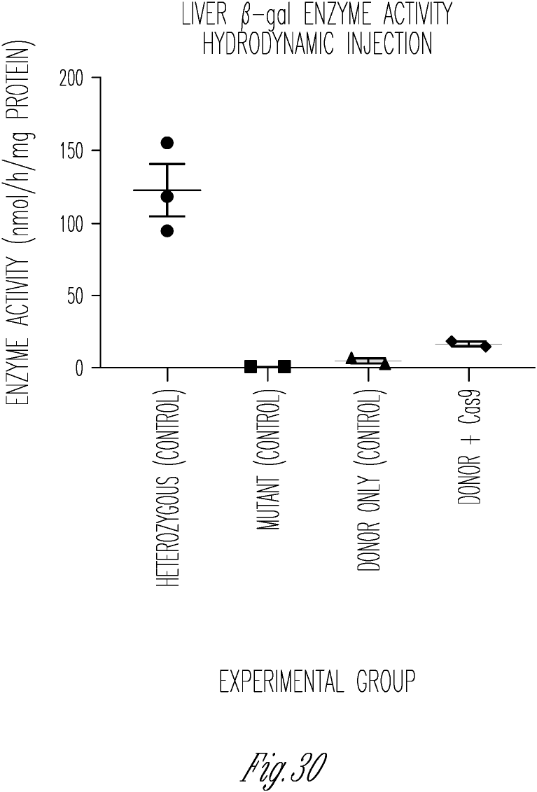

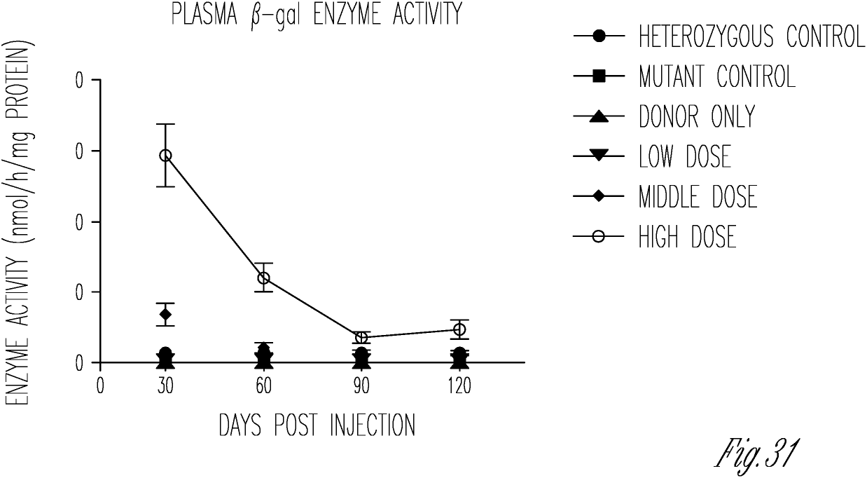

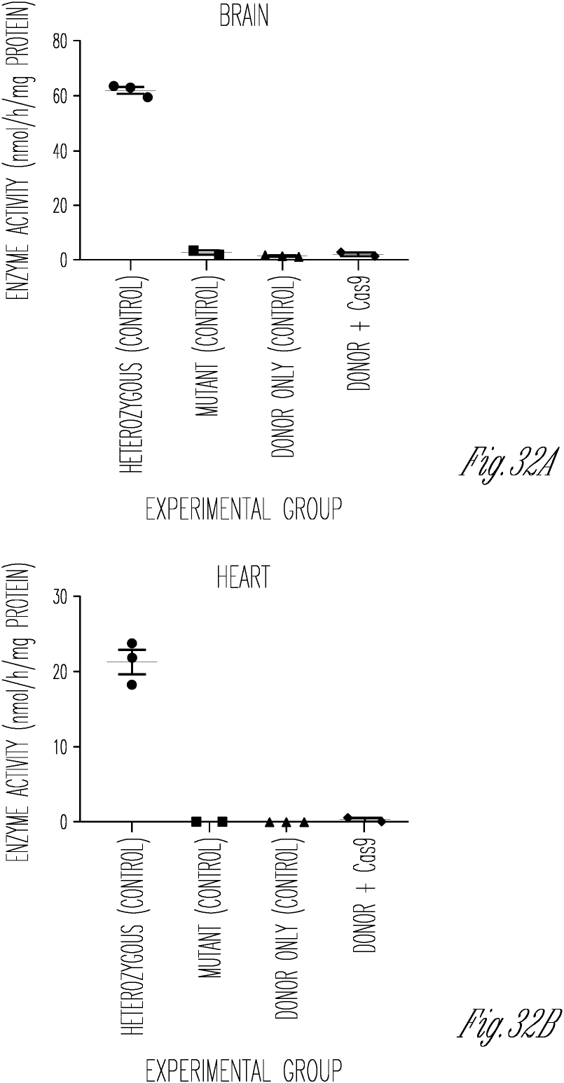

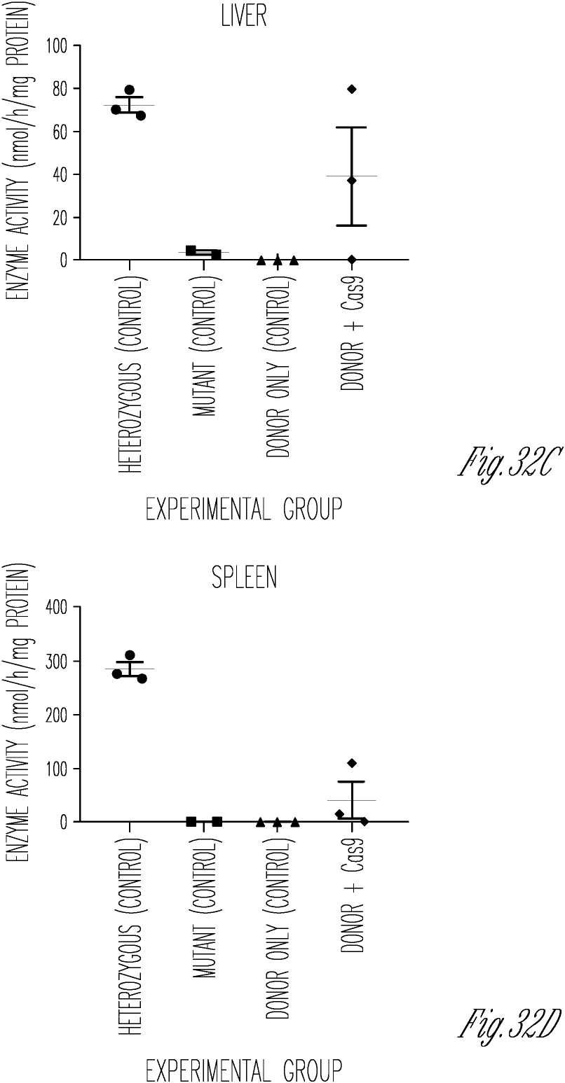

### DETAILED DESCRIPTION

### Definitions

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

The term “disease” or “disorder” are used interchangeably, and are used to refer to diseases or conditions wherein lack of or reduced amounts of a specific gene product, e.g., a lysosomal storage enzyme, plays a role in the disease such that a therapeutically beneficial effect can be achieved by supplementing, e.g., to at least 1% of normal levels.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent, e.g., a recombinant AAV encoding a gene product, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result. A therapeutically effective amount is also one in which any toxic or detrimental effects of the agent(s)are outweighed by the therapeutically beneficial effects.

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest) and/or a selectable or detectable marker.

“AAV” is adeno-associated virus, and may be used to refer to the virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on its binding properties, e.g., there are eleven serotypes of AAVs, AAV1-AAV11, including AAV2, AAV5, AAV6, AAV8, AAV9 and AAVrh10, and the term encompasses pseudotypes with the same binding properties. Thus, for example, AAV9 serotypes include AAV with the binding properties of AAV9, e.g., a pseudotyped AAV comprising AAV9 capsid and a rAAV genome which is not derived or obtained from AAV9 or which genome is chimeric. The abbreviation “rAAV” refers to recombinant adeno-associated virus, also referred to as a recombinant AAV vector (or “rAAV vector”).

An “AAV virus” refers to a viral particle composed of at least one AAV capsid protein and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”. An AAV “capsid protein” includes a capsid protein of a wild-type AAV, as well as modified forms of an AAV capsid protein which are structurally and or functionally capable of packaging a rAAV genome and bind to at least one specific cellular receptor which may be different than a receptor employed by wild type AAV. A modified AAV capsid protein includes a chimeric AAV capsid protein such as one having amino acid sequences from two or more serotypes of AAV, e.g., a capsid protein formed from a portion of the capsid protein from AAV9 fused or linked to a portion of the capsid protein from AAV-2, and a AAV capsid protein having a tag or other detectable non-AAV capsid peptide or protein fused or linked to the AAV capsid protein, e.g., a portion of an antibody molecule which binds a receptor other than the receptor for AAV9, such as the transferrin receptor, may be recombinantly fused to the AAV9 capsid protein.

A “pseudotyped” rAAV is an infectious virus having any combination of an AAV capsid protein and an AAV genome. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 inverted terminal repeats (ITRs), each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the invention may be homologous, i.e., from the same serotype, heterologous, i.e., from different serotypes, or chimeric, i.e., an ITR which has ITR sequences from more than one AAV serotype.

The terms “nucleic acid,” “polynucleotide,” and “oligonucleotide” are used interchangeably and refer to a deoxyribonucleotide or ribonucleotide polymer, in linear or circular conformation, and in either single-or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. “Affinity” refers to the strength of binding: increased binding affinity being correlated with a lower Kd.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

A “disease associated gene” is one that is defective in some manner in a monogenic disease. Non-limiting examples of monogenic diseases include severe combined immunodeficiency, cystic fibrosis, lysosomal storage diseases (e.g. Gaucher’s, Hurler’s Hunter’s, Fabry’s, Neimann-Pick, Tay-Sach’s etc), sickle cell anemia, and thalassemia.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single-or double-stranded; can be linear, branched or circular; and can be of any length. Nucleic acids include those capable of forming duplexes, as well as triplex-forming nucleic acids.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (e.g., liposomes, including neutral and cationic lipids), electroporation, direct injection, cell fusion, particle bombardment, calcium phosphate coprecipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence.

### The CRISPR/Cas System

The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system. The primary products of the CRISPR loci appear to be short RNAs that contain the invader targeting sequences, and are termed guide RNAs

“Cas1” polypeptide refers to CRISPR associated (Cas) protein1. Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cas1 polypeptide is a Cas1 polypeptide of a bacterium. In certain embodiments, a Cas1 polypeptide is a Cas1 polypeptide of a gram negative or gram positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex aeolicus. In certain embodiments, a Cas1 polypeptide is a Cas1 polypeptide that is a member of one of CASs1-7. In certain embodiments, Cas1 polypeptide is a Cas1 polypeptide that is a member of CASS3. In certain embodiments, a Cas1 polypeptide is a Cas1 polypeptide that is a member of CASS7. In certain embodiments, a Cas1 polypeptide is a Cas1 polypeptide that is a member of CASS3 or CASS7.

In some embodiments, a Cas1 polypeptide is encoded by a nucleotide sequence provided in GenBankat, e.g., GenelD number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cas1 polypeptides.

There are three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif)). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA (see Jinek, et al. (2012) Science 337:816 and Cong et al. (2013)

Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA

“Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof.

### RNA Components of CRISPR/Cas

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong, et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek, ibid and Cong, ibid).

Chimeric or sgRNAs can be engineered to comprise a sequence complementary to any desired target. The RNAs comprise 22 bases of complementarity to a target and of the form G[n19], followed by a protospacer-adjacent motif (PAM) of the form NGG. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence that conforms to the G[n20]GG formula. Donors

As noted above, insertion of an exogenous sequence (also called a “donor sequence” or “donor” or “transgene” or “gene of interest”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Alternatively, a donor may have no regions of homology to the targeted location in the DNA and may be integrated by NHEJ-dependent end joining following cleavage at the target site. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang, et al. (1987) Proc. Natl. Acad. Sci. USA 84:4959-4963; Nehls, et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted (e.g., highly expressed, albumin, AAVS1, HPRT, etc.). However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an albumin or other locus such that some (N-terminal and/or C-terminal to the transgene encoding the lysosomal enzyme) or none of the endogenous albumin sequences are expressed, for example as a fusion with the transgene encoding the lysosomal sequences. In other embodiments, the transgene (e.g., with or without additional coding sequences such as for albumin) is integrated into any endogenous locus, for example a safe-harbor locus. See, e.g., U.S. Pat. Publication Nos. 2008/0299580; 2008/0159996; and 2010/0218264.

When endogenous sequences (endogenous or part of the transgene) are expressed with the transgene, the endogenous sequences (e.g., albumin, etc.) may be full-length sequences (wild-type or mutant) or partial sequences. Preferably the endogenous sequences are functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

### Exemplary rAAV Vectors

Adeno-associated viruses of any serotype are suitable to prepare rAAV, since the various serotypes are functionally and structurally related, even at the genetic level. All AAV serotypes apparently exhibit similar replication properties mediated by homologous rep genes; and all generally bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control. Among the various AAV serotypes, AAV2 is most commonly employed.

An AAV vector of the invention typically comprises a polynucleotide that is heterologous to AAV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and may be preferred when it is desired that the therapeutic or prophylactic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will preferably also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, e.g., (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present invention.

The native promoters for rep are self-regulating, and can limit the amount of AAV particles produced. The rep gene can also be operably linked to a heterologous promoter, whether rep is provided as part of the vector construct, or separately. Any heterologous promoter that is not strongly downregulated by rep gene expression is suitable; but inducible promoters may be preferred because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1 CP4; as well as vaccinia or poxvirus inducible promoters.

Methods for identifying and testing helper-virus-inducible promoters have been described (see, e.g., WO 96/17947). Thus, methods are known in the art to determine whether or not candidate promoters are helper-virus-inducible, and whether or not they will be useful in the generation of high efficiency packaging cells. Briefly, one such method involves replacing the p5 promoter of the AAV rep gene with the putative helper-virus-inducible promoter (either known in the art or identified using well-known techniques such as linkage to promoter-less “reporter” genes). The AAV rep-cap genes (with p5 replaced), e.g., linked to a positive selectable marker such as an antibiotic resistance gene, are then stably integrated into a suitable host cell (such as the HeLa or A549 cells exemplified below). Cells that are able to grow relatively well under selection conditions (e.g., in the presence of the antibiotic) are then tested for their ability to express the rep and cap genes upon addition of a helper virus. As an initial test for rep and/or cap expression, cells can be readily screened using immunofluorescence to detect Rep and/or Cap proteins. Confirmation of packaging capabilities and efficiencies can then be determined by functional tests for replication and packaging of incoming rAAV vectors. Using this methodology, a helper-virus-inducible promoter derived from the mouse metallothionein gene has been identified as a suitable replacement for the p5 promoter, and used for producing high titers of rAAV particles (as described in WO 96/17947).

Removal of one or more AAV genes is in any case desirable, to reduce the likelihood of generating replication-competent AAV (“RCA”). Accordingly, encoding or promoter sequences for rep, cap, or both, may be removed, since the functions provided by these genes can be provided in trans, e.g., in a stable line or via co-transfection.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this invention in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. 5,658,776). Alternatively, a stable mammalian cell line with an AAV rep gene operably linked to a promoter can be used to supply replication functions (see, e.g., Trempe et al., WO 95/13392); Burstein et al. (WO 98/23018); and Johnson et al. (U.S. No. 5,656,785). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this invention.

### Compositions and Routes of Delivery

Any route of administration may be employed so long as that route and the amount administered are prophylactically or therapeutically useful.

In vivo administration of the components, e.g., delivered in a viral vector such as a lentivirus or AAV vector, and compositions containing them, can be accomplished by any suitable method and technique presently or prospectively known to those skilled in the art. The subject polynucleotides or polypeptides can be formulated in a physiologically- or pharmaceutically-acceptable form and administered by any suitable route known in the art including, for example, oral, nasal, rectal, transdermal, vaginal, and parenteral routes of administration. As used herein, the term parenteral includes subcutaneous, intradermal, intravenous, intramuscular, intraperitoneal, and intracisternal administration, such as by injection.

Administration of the compositions can be a single administration, or at continuous or distinct intervals as can be readily determined by a person skilled in the art. In one embodiment, a polynucleotide component is stably incorporated into the genome of a person of animal in need of treatment. Methods for providing gene therapy are well known in the art.

The compositions can also be administered utilizing liposome and nano-technology, slow release capsules, implantable pumps, and biodegradable containers, and orally or intestinalily administered intact plant cells expressing the therapeutic product. These delivery methods can, advantageously, provide a uniform dosage over an extended period of time.

Suitable dose ranges for are generally about 10³ to 10¹⁵ infectious units of viral vector per microliter delivered in 1 to 3000 microliters of single injection volume. For instance, viral genomes or infectious units of vector per micro liter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or10¹⁷ viral genomes or infectious units of viral vector delivered in about 10, 50, 100, 200, 500, 1000, or 2000 microliters. It should be understood that the aforementioned dosage is merely an exemplary dosage and those of skill in the art will understand that this dosage may be varied. Effective doses may be extrapolated from dose-responsive curves derived from in vitro or in vivo test systems.

In one embodiment, suitable dose ranges are generally about 10³ to 10¹⁵ infectious units of viral vector per microliter delivered in, for example, 1, 2, 5, 10, 25, 50, 75 or 100 or more milliliters, e.g.,1 to 10,000 milliliters or 0.5 to 15 milliliters, of single injection volume. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, or 10¹⁴ viral genomes or infectious units of viral vector. In one embodiment, suitable dose ranges, generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, 10¹⁴, 110¹⁵, 10¹⁶, or 10¹⁷ viral genomes or infectious units of viral vector, e.g., at least 1.2 x 10¹¹ genomes or infectious units, for instance at least 2 x 10¹¹ up to about 2 x 10¹² genomes or infectious units or about 1 x 10¹² to about 5 ×10¹⁶ genomes or infectious units..

Administration of agents in accordance with the present invention can be achieved by direct injection of the composition or by the use of infusion pumps. For injection, the composition can be formulated in liquid solutions, e.g., in physiologically compatible buffers such as Hank’s solution, Ringer’s solution or phosphate buffer. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

In one embodiment, the agent(s) may be administered by any route including parenterally. In one embodiment, the agent(s) may be administered by subcutaneous, intramuscular, or intravenous injection, orally, intrathecally, or intracranially, or by sustained release, e.g., using a subcutaneous implant. The the agent(s) may be dissolved or dispersed in a liquid carrier vehicle. For parenteral administration, the active material may be suitably admixed with an acceptable vehicle, e.g., of the vegetable oil variety such as peanut oil, cottonseed oil and the like. Other parenteral vehicles such as organic compositions using solketal, glycerol, formal, and aqueous parenteral formulations may also be used. For parenteral application by injection, the agent(s) may comprise an aqueous solution of a water soluble pharmaceutically acceptable salt of the active acids according to the invention, desirably in a concentration of 0.01-10%, and optionally also a stabilizing agent and/or buffer substances in aqueous solution. Dosage units of the solution may advantageously be enclosed in ampules.

The agent(s) may be in the form of an injectable unit dose. Examples of carriers or diluents usable for preparing such injectable doses include diluents such as water, ethyl alcohol, macrogol, propylene glycol, ethoxylated isostearyl alcohol, polyoxyisostearyl alcohol and polyoxyethylene sorbitan fatty acid esters, pH adjusting agents or buffers such as sodium citrate, sodium acetate and sodium phosphate, stabilizers such as sodium pyrosulfite, EDTA, thioglycolic acid and thiolactic acid, isotonic agents such as sodium chloride and glucose, local anesthetics such as procaine hydrochloride and lidocaine hydrochloride. Furthermore, usual solubilizing agents and analgesics may be added. injections can be prepared by adding such carriers to the enzyme or other active, following procedures well known to those of skill in the art. A thorough discussion of pharmaceutically acceptable excipients is available in REMINGTON’S PHARMACEUTICAL SCIENCES (Mack Pub. Co., N.J. 1991). The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

When the agent(s) is administered in the form of a subcutaneous implant, the compound is suspended or dissolved in a slowly dispersed material known to those skilled in the art, or administered in a device which slowly releases the active material through the use of a constant driving force such as an osmotic pump. In such cases, administration over an extended period of time is possible.

The dosage at which the agent(s) is administered may vary within a wide range and will depend on various factors such as the severity of the disease, the age of the patient, etc., and may have to be individually adjusted. Compositions described herein may be employed in combination with another medicament. The compositions can appear in conventional forms, for example, aerosols, solutions, suspensions, or topical applications, or in lyophilized form.

Typical compositions include the agent(s) and a pharmaceutically acceptable excipient which can be a carrier or a diluent. For example, the active agent(s) may be mixed with a carrier, or diluted by a carrier, or enclosed within a carrier. When the active agent is mixed with a carrier, or when the carrier serves as a diluent, it can be solid, semi-solid, or liquid material that acts as a vehicle, excipient, or medium for the active agent. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active agent(s). Such additives can include wetting agents, emulsifying and suspending agents, salt for influencing osmotic pressure, buffers and/or coloring substances preserving agents, sweetening agents or flavoring agents. The compositions can also be sterilized if desired.

If a liquid carrier is used, the preparation can be in the form of a liquid such as an aqueous liquid suspension or solution. Acceptable solvents or vehicles include sterilized water, Ringer’s solution, or an isotonic aqueous saline solution.

The agent(s) may be provided as a powder suitable for reconstitution with an appropriate solution as described above. Examples of these include, but are not limited to, freeze dried, rotary dried or spray dried powders, amorphous powders, granules, precipitates, or particulates. The composition can optionally contain stabilizers, pH modifiers, surfactants, bioavailability modifiers and combinations of these. A unit dosage form can be in individual containers or in multi-dose containers.

Compositions contemplated by the present invention may include, for example, micelles or liposomes, or some other encapsulated form, or can be administered in an extended release form to provide a prolonged storage and/or delivery effect, e.g., using biodegradable polymers, e.g., polylactide-polyglycolide. Examples of other biodegradable polymers include poly(orthoesters) and poly(anhydrides).

Polymeric nanoparticles, e.g., comprised of a hydrophobic core of polylactic acid (PLA) and a hydrophilic shell of methoxy-poly(ethylene glycol) (MPEG), may have improved solubility and targeting to the CNS. Regional differences in targeting between the microemulsion and nanoparticle formulations may be due to differences in particle size.

Liposomes are very simple structures consisting of one or more lipid bilayers of amphiphilic lipids, i.e., phospholipids or cholesterol. The lipophilic moiety of the bilayers is turned towards each other and creates an inner hydrophobic environment in the membrane. Liposomes are suitable drug carriers for some lipophilic drugs which can be associated with the non-polar parts of lipid bilayers if they fit in size and geometry. The size of liposomes varies from 20 nm to few µm.

Mixed micelles are efficient detergent structures which are composed of bile salts, phospholipids, tri, di- and monoglycerides, fatty acids, free cholesterol and fat soluble micronutrients. As long-chain phospholipids are known to form bilayers when dispersed in water, the preferred phase of short chain analogues is the spherical micellar phase. A micellar solution is a thermodynamically stable system formed spontaneously in water and organic solvents. The interaction between micelles and hydrophobic/lipophilic drugs leads to the formation of mixed micelles (MM), often called swallen micelles, too. In the human body, they incorporate hydrophobic compounds with low aqueous solubility and act as a reservoir for products of digestion, e.g. monoglycerides.

Lipid microparticles includes lipid nano- and microspheres. Microspheres are generally defined as small spherical particles made of any material which are sized from about 0.2 to 100 µm. Smaller spheres below 200 nm are usually called nanospheres. Lipid microspheres are homogeneous oil/water microemulsions similar to commercially available fat emulsions, and are prepared by an intensive sonication procedure or high pressure emulsifying methods (grinding methods). The natural surfactant lecithin lowers the surface tension of the liquid, thus acting as an emulsifier to form a stable emulsion. The structure and composition of lipid nanospheres is similar to those of lipid microspheres, but with a smaller diameter.

Polymeric nanoparticles serve as carriers for a broad variety of ingredients. The active components may be either dissolved in the polymetric matrix or entrapped or adsorbed onto the particle surface. Polymers suitable for the preparation of organic nanoparticles include cellulose derivatives and polyesters such as poly(lactic acid), poly(glycolic acid) and their copolymer. Due to their small size, their large surface area/volume ratio and the possibility of functionalization of the interface, polymeric nanoparticles are ideal carrier and release systems. If the particle size is below 50 nm, they are no longer recognized as particles by many biological and also synthetic barrier layers, but act similar to molecularly disperse systems.

Thus, the composition of the invention can be formulated to provide quick, sustained, controlled, or delayed release, or any combination thereof, of the active agent after administration to the individual by employing procedures well known in the art. In one embodiment, the enzyme is in an isotonic or hypotonic solution. In one embodiment, for enzymes that are not water soluble, a lipid based delivery vehicle may be employed, e.g., a microemulsion such as that described in WO 2008/049588, the disclosure of which is incorporated by reference herein, or liposomes.

In one embodiment, the preparation can contain an agent, dissolved or suspended in a liquid carrier, such as an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

### Exemplary Diseases

The composition(s) may be employed to prevent, inhibit or treat monogenic diseases including but not limited to lysosomal storage diseases, hemophilia, e.g., lack of or decreased factor VIII or IX production, sickle cell disease and thalassemia, e.g., lack of beta-globin or alpha-globin production. Lysosomal diseases and (parenthetically) related enzymes and proteins associated with diseases that are contemplated within the scope of the invention include, but are not limited to, Activator Deficiency/GM2 Gangliosidosis (beta-hexosaminidase), Alpha-mannosidosis (alpha-D-mannosidase), Aspartylglucosaminuria (aspartylglucosaminidase), Cholesteryl ester storage disease (lysosomal acid lipase), Chronic Hexosaminidase A Deficiency (hexosaminidase A), Cystinosis (cystinosin), Danon disease (LAMP2), Fabry disease (alpha-galactosidase A), Farber disease (ceramidase), Fucosidosis (alpha-L-fucosidase), Galactosialidosis (cathepsin A), Gaucher Disease (Type I, Type II, Type III) (beta-glucocerebrosidase), GM1 gangliosidosis (Infantile, Late infantile/Juvenile, Adult/Chronic) (beta-galactosidase), I-Cell disease/Mucolipidosis II (GioNAc-phosphotransferase), Infantile Free Sialic Acid Storage Disease/ISSD (sialin), Juvenile Hexosaminidase A Deficiency ((hexosaminidase A), Krabbe disease (Infantile Onset, Late Onset) (galactocerebrosidase), Metachromatic Leukodystrophy (arylsulfatase A), Mucopolysaccharidoses disorders [Pseudo-Hurler polydystrophy/Muco lipidosis IIIA (N-acetylglucosamine-1 -phosphotransferase), MPSI Hurler Syndrome (alpha-L iduronidase), MPSI Scheie Syndrome (alpha-L iduronidase), MPS I Hurler-Scheie Syndrome (alpha-L iduronidase), MPS II Hunter syndrome (iduronate-2-sulfatase), Sanfilippo syndrome Type A/MPS III A (heparan N-sulfatase), Sanfilippo syndrome Type B/MPS III B (N-acetyl-alpha-D-glucosaminidase), Sanfilippo syndrome Type C/MPS III C (acetyl-CoA, alpha-glucosaminide acetyltransferase, Sanfilippo syndrome Type D/fvlPS III D (N-acetylglucosamine-G-sulfate-sulfatase), Morquio Type A/MPS IVA (N-acetylgalatosamine-6-sulfate-sulfatase), Morquio Type B/MPS IVB (β-galactosidase-I), MPS IX Hyaluronidase Deficiency (hyaluronidase), MPS VI Maroteaux-Lamy (arylsulfatase B), MPS VII Sly Syndrome (beta-glucuronidase), Mucolipidosis I/Sialidosis (alpha-N -acetyl neuraminidase), Mucolipidosis IIIC (N-acetylglucosamine-1 -phosphotransferase), Mucolipidosis type IV (mucolipinl)], Multiple sulfatase deficiency (multiple sulfatase enzymes), Niemann-Pick Disease (Type A, Type B, Type C) (sphingomyelinase), Neuronal Ceroid Lipofuscinoses [(CLN6 disease - Atypical Late Infantile, Late Onset variant, Early Juvenile (ceroid-lipofuscinosis neuronal protein 6); Batten-Spielmeyer-Vogt/Juvenile NCL/CLN3 disease (battenin); Finnish Variant Late Infantile CLN5 (ceroid-lipofuscinosis neuronal protein 5); Jansky-Bielschowsky disease/Late infantile CLN2/TPP1 Disease (tripeptidyl peptidase 1); Kufs/ Adult-onset NCL/CLN4 disease; Northern Epilepsy/variant late infantile CLN8 (ceroid-lipofuscinosis neuronal protein 8); Santavuori-Haltia/Infantile CLN1/PPT disease (palmitoyl-protein thioesterase 1); Beta-mannosidosis (beta-mannosidase)], Tangier disease (ATP-binding cassette transporter ABCAI), Pompe disease/Glycogen storage disease type II (acid maltase), Pycnodysostosis (cathepsin K), Sandhoff disease/ Adult Onset/GM2 Gangliosidosis (beta-hexosaminidases A and B), Sandhoff disease/GM2 gangliosidosis - Infantile, Sandhoff disease/GM2 garigliosidosis - Juvenile (beta-hexosaminidases A and B), Schindler disease (alpha-N-acetylgalactosaminidas), Salla disease/Sialic Acid Storage Disease (sialin), Tay-Sachs/GM2 gangliosidosis (beta-hexosaminidase), and Wolman disease (lysosomal acid lipase), Sphingolipidosis, Hurrnansky-Pudiak Syndrome (HPS1, HPS3, HPS4, HPS5, HPS6 and HPS7) Type 2 - AP-3 complex subunit beta-1, Type 7 -dysbindin), Chediak-Higashi Syndrome (lysosomal trafficking regulator protein), and Griscelli disease (Type 1 : myosin-Va, Type 2: ras-related protein Rab-27A, Type 3: melanophilin).

Additional diseases (including related proteins) include the neurodegenerative diseases which include but are not limited to Parkinson’s, Alzheimer’s, Huntington’s, and Amyotrophic Lateral Sclerosis ALS (superoxide dismutase), Hereditary emphysema (a 1 -Antitrypsin), Oculocutaneus albinism (tyrosinase), Congenital sucrase-isomaltase deficiency (Sucrase-isomaltase), and Choroideremia (Repl) Lowe’s Oculoceribro-renal syndrome (PIP2-5-phosphatase).

In one embodiment, the disorder or disease is Activator Deficiency/GM2 Gangliosidosis, Alpha-mannosidosis, Aspartylglucosaminuria, Cholesteryl ester storage disease, Chronic Hexosaminidase A Deficiency, Cystinosis, Danon disease, Fabry disease, Farber disease, Fucosidosis, Galactosialidosis, Gaucher Disease (Type I, Type II, Type III), GM1 gangliosidosis (Infantile, Late infantile/Juvenile, Adult/Chronic), I-Cell disease/Mucolipidosis II, Infantile Free Sialic Acid Storage Disease/ISSD, Juvenile Hexosaminidase A Deficiency, Krabbe disease (Infantile Onset, Late Onset), Metachromatic Leukodystrophy, Mucopolysaccharidoses disorders (Pseudo-Hurler polydystrophy/Mucolipidosis IIIA, MPSI Hurler Syndrome, MPSI Scheie Syndrome, MPS I Hurler-Scheie Syndrome, MPS II Hunter syndrome, Sanfilippo syndrome Type A/MPS III A, Sanfilippo syndrome Type B/MPS III B, Sanfilippo syndrome Type C/MPS III C, Sanfilippo syndrome Type D/MPS III D, Morquio Type A/MPS IVA, Morquio Type B/MPS IVB, MPS IX Hyaluronidase Deficiency, MPS VI Maroteaux-Lamy, MPS VII Sly Syndrome, Mucolipidosis I/Sialidosis, Mucolipidosis IIIC, Mucolipidosis type IV), Multiple sulfatase deficiency, Niemann-Pick Disease (Type A, Type B, Type C), Neuronal Ceroid Lipofuscinoses (CLN6 disease -Atypical Late Infantile, Late Onset variant, Early Juvenile; Batten-Spielmeyer-Vogt/Juvenile NCL/CLN3 disease; Finnish Variant Late Infantile CLN5; Jansky-Bielschowsky disease/Late infantile CLN2/TPP1 Disease; Kufs/ Adult-onset NCL/CLN4 disease; Northern Epilepsy/variant late infantile CLN8; Santavuori-Haltia/lnfantile CLN1 /PPT disease; Beta-mannosidosis), Tangier disease, Pompe disease/Glycogen storage disease type II, Pycnodysostosis, Sandhoff disease/Adult Onset/GM2 Gangliosidosis, Sandhoff disease/GM2 gangliosidosis - Infantile, Sandhoff disease/GM2 gangliosidosis - Juvenile, Schindler disease, Salla disease/Sialic Acid Storage Disease, Tay-Sachs/GM2 gangliosidosis, Wolman disease, Sphingolipidosis, Hurmansky-Pudiak Syndrome, Chediak-Higashi Syndrome, or Griscelli disease.

The invention will be described by the following non-limiting examples.

### Example 1

Gene therapy holds promise for treating lysosomal diseases as it has potential for permanent, single-dose treatment. Currently, treatment protocols providing sustained therapeutic benefits with minimized safety risks for patients with lysosomal diseases are in desperate need. To this end, two constructs were designed: one encoding Cas9 targeting intron 1 of albumin locus, and the other encoding promoterless IDUA cDNA sequence. A total of four guide RNAs (gRNAs) were designed and transfected into fibroblast cells together with SaCas9. The ability of these gRNAs to guide Cas9-mediated cleavage at the albumin locus was evaluated via the Surveyor assay. Two days after hydrodynamic injection of these two plasmids into MPS I mice, only the mice receiving both plasmids (n=3) had significant higher IDUA enzyme activities in liver (2.7 fold of wildtype levels). Mice receiving the plasmid encoding promoterless cDNA donor (n=3) had no increase in IDUA activity. Deep sequencing showed that the %indels at the target locus was only 0.2%, which yielded substantial enzyme expression in 2 days. To further evaluate this strategy, the two constructs were packaged into AAV8 vectors, and were injected into neonatal MPS I mice at different doses. To determine the efficacy, IDUA enzyme activities and GAG levels are measured, neurocognitive behaviors are assessed, and cellular vacuolation is evaluated by electron microscopy. Moreover, on-target and off-target gene modification rates, are assessed, residual Cas9 activity determined and vector copy number quantified. Results from this study are applicable for a clinical protocol of CRISPR-mediated in vivo genome editing to treat patients such as those with lysosomal storage disorders, mucoploysaccharidoses, e.g., MPS I patients, and blood disorders including hemophilia and thalassemia.

In a previous study with zinc finger nucleases (ZFNs), approximately 0.5% of mRNA from albumin locus was the fusion transcript, indicating a relatively low genome modification rate likely due to the use of 3 AAV vectors for transduction of a single hepatocyte. For humans, a higher dose may be needed and a higher dose brings about higher rates of off-target effects, more challenge for vector production and higher manufacturing costs. The CRISPR (Clustered Regulatory interspaced Short Palindromic Repeats) system emerges as a powerful alternative because of its high targeting efficiency and ease of design. A new Cas9 ortholog, Staphylococcus aureus Cas9 (SaCas9), that is short enough to fit into AAV vectors, has been reported (Ran et al., 2015). in this study, no off-target events were observed in the mice after AAV delivery of SaCas9 and guide RNAs. More interestingly, three independent gene therapy studies using SaCas9 observed undetectable (Yang et al., 2016) or minimal (Nelson et al., 2016; Tabebordbar et al., 2016) off-target effects, indicating a very high specificity. Considering the high efficiency and specificity, a Cas based system, e.g., SaCas9, delivered by vectors including viral vectors, e.g., AAV vectors, was used. As opposed to 3 AAV vectors used in the study with ZFNs, this CRISPR/Cas system has 1 or 2 vectors. For the 2 vector system, in one embodiment, one vector encodes Cas9 and guide RNA, and the other encodes a promoterless donor sequence; in another embodiment, one vector encodes Cas9 and the other vector encodes the promoterless donor sequence and guide RNA. Assuming similar doses when using rAAV, and similar AAV transduction and nuclease targeting efficiency, the efficiency of successful genome editing by CRISPR is higher. Thus, such as CRISPR-mediated genome editing strategy may allow for the use of lower dose sof AAV vectors for treating diseases including lysosomal diseases, which brings minimized risk, ease of vector production and less expense.

The design for CRISPR-mediated in vivo genome editing for MPS I mice includes, in one embodiment, i.v. administration of 2 different AAV vectors (AAV8 encoding Cas and gRNA, AA V8 carrying promoterless IDUA cDNA). With AAV carrying IDUA sequence and flanking homology sequences, IDUA sequence was inserted into albumin locus e through homology-directed repair (HDR). The splicing donor sequence at exon 1 of albumin locus interacted with the splicing acceptor preceding the donor sequence. Therefore, under control of the endogenous albumin promoter, a fusion transcript of albumin exon 1 and IDUA was generated. Since exon 1 of albumin mainly encodes signal peptide and was cleaved thereafter, the mature protein was IDUA enzyme only.

Cas9, e.g., SaCas9, and guide RNA can also mediate the insertion of HEXB cDNA into albumin locus and achieve expression of Hex enzyme. AAV8 vectors are liver-tropic, and SaCas9 is under control of a liver-specific promoter. By virtue of this, genome editing and transgene expression can be limited to hepatocytes. Systemic therapeutic benefits zfd achieved through a phenomenon called ‘cross correction’. A total of four guide RNAs (gRNAs) were designed and transfected into fibroblast cells together with SaCas9. The ability of these gRNAs to guide SaCas9-mediated cleavage at the albumin locus was evaluated via the SURVERYOR assay. The results showed that one of the gRNAs, g1 (5′GTATCTTTGATGACAATAATGGGGGAT3′; SEQ ID NO:3) mediated targeted DNA cleavage with the highest efficiency (11% indels, and was selected for future studies). Plasmids encoding SaCas9 and IDUA cDNA donor in MPS I mice through hydrodynamic injection. Only the mice receiving both plasmids had significant higher IDUA enzyme activities in liver (2.7 fold of wildtype levels). Mice receiving the plasmid encoding promoterless cDNA donor had no increase in IDUA activities. These results strongly support the feasibility of this CRISPR-mediated safe harbor genome editing strategy in treating MPS I mice.

### Example 2

In order to establish a gene therapy protocol to achieve a satisfactory clinical outcome or good quality of life for patients with MPS I and other lysosomal diseases, a genome editing protocol which can provide sustained therapeutic benefits multiple tissues including the brain, and minimize the vector-associated risk was tested. A single administration of AAV vectors delivering the CRISPR system targeting, for example, the albumin locus of hepatocyte, may treat both systemic and neurological diseases of MPS I with minimized risks. The feasibility of this study is supported by preliminary data. As described herein, co-delivery of 2 AAV vectors, one of which a promoterless IDUA cDNA donor can efficiently facilitate insertion of IDUA sequence into the albumin locus through homology directed repair (HDR). The endogenous albumin promoter drives IDUA transgene expression, which is likely sufficient to treat both systemic and neurological diseases of MPS I through cross correction.

AAV delivery of the CRISPR system for genome editing in neonatal MPS I mice. Neonatal gene therapy can enhance enzyme delivery to tissues including the brain due to the naive immune system and relatively permeable blood-brain-barrier in the neonatal period (Hinderer et al.,2015). To test this CRISPR-mediated genome editing strategy in neonatal mice, newborn MPS I pups are i.v. administered with a dual AAV system (AAV8-SaCas9-sgRNA and AAV8-IDUA) through temporal facial vein. To determine the efficacy, IDUA transgene expression and GAG storage levels in tissues are measured, and behavior tests are conducted. Gene modification events are analyzed, vector biodistribution is determined, and tumorigenesis risk is assessed by pathological analysis.

Neonatal mice are used for three main reasons. (1) Since newborn pups (~1 g) need substantially less vector, it could function as a dosing-finding study before producing large amount of vectors for adult mice (~25 g). (2) It has been shown that neonatal administration of AAV vectors can induce immune tolerance and improve the safety and efficacy of gene therapy (Hinderer et al., 2015). (3) Since the implementation of newborn screening for MPS I (Scott et al., 2013) enables very early treatment of patients, it is essential to evaluate this genome editing strategy in neonatal mice. In summary, this data can be extrapolated into a clinical protocol for treating human babies with MPS I.

The effects of in utero genome editing mediated by the CRISPR system. Preliminary data showed glycosaminoglycan inclusions at postcoital day 14 (E14) in MPS I mice. To test the working hypothesis that prenatal treatment can prevent irreversible damage, the same dual AAV system is administered via intrahepatic injection at E14. Treated mice are evaluated as described above. In addition, since AAV vectors can cross the placenta (Mattar et al., 2011), to monitor the safety profile, vector biodistribution and gene modification events in the dams are determined.

AAV delivery of the CRISPRsystem for genome editing in adult MPS I mice. To determine the extent to which robust liver growth in neonates is essential for therapeutic levels of genome editing, the CRISPR-mediated genome editing strategy is tested in adult MPS I mice. Immune tolerization is conducted through administration of IDUA proteins starting from the neonatal stage. Adult MPS I mice are i.v. administered with the same dual AAV system through tail vein. Treated mice are analyzed as described above. Additionally, the treatment effects on proteomics and metabolomics profiles of MPS I mice are determined.

The use of the CRISPR system will likely result in high levels of IDUA in treated MPS I mice, normalizing GAG accumulation and providing neurological This study will, for the first time, evaluate safety and efficacy of in vivo delivery of CRISPR by AAV to edit hepatocytes and thus treat MPS I.

### Strategy

Mucopolysaccharidosis type I (MPS I) is an autosomal recessive disease that results from deficiency of α-L-iduronidase (IDUA), and subsequent accumulation of glycosaminoglycans (GAG). MPS I leads to coarse facial feature, growth delay, organomegaly, progressive neurodegeneration, mental retardation and death before the age of 10 (Neufeld et al., 2001). Currently, MPS I patients are treated by enzyme replacement therapy (ERT) and hematopoietic stem cell transplantation (HSCT). However, ERT is of limited use due to the need for frequent, life long, expensive (>$200,000 annually) treatments, and negligible neurological benefits (Wraith et al., 2004). HSCT can lead to prolonged survival (Moore et al., 2008), somatic improvements and partial neurological benefits (Prasad et al., 2008), but is associated with morbidity or mortality (Boelens et al., 2009). Multiple preclinical gene therapy studies for treating MPS I using retroviral (Traas 2007), lentiviral vectors (e.g., Di Domenico et al., 2006) and adeno-associated virus (AAV) vectors (e.g., Wolf et al., 2011) have been conducted. However, the lentiviral and retroviral vectors mainly rely on random integration, which poses risk of insertional mutagenesis leading to cancer and germline transmission. The direct evidence comes from the clinical trial for treating X-linked severe combined immunodeficiency with retroviral gene therapy, 2 patients developed leukemia after treatment due to oncogene activation by retroviral integration (Hacein-Bey-Abina et al., 2003). Further, although AAV can integrate itself into host genome at a very low frequency (Nakai et al., 2003), AAV is mainly an episomal vector which is not expected to provide long-term transgene expression. Evidence comes from a study showing that after one round of cell division, transgene expression from episomal AAV vectors was rapidly lost (Nakai et al., 2001). Secondary administration of an AAV vector will be unlikely to achieve successful transduction, due to immune responses resulting from primary vector administration (Calcedo et al., 2013). Collectively, affordable treatment protocols providing sustained therapeutic benefits with minimized safety risks for MPS I patients are in desperate need.

### In Vivo Genome Editing of Albumin Locus to Treat MPS I

Genome editing emerges as a promising approach because it enables long-term transgene expression and minimizes insertional mutagenesis risk due to random integration. A standard genome editing approach is to repair the disease-causing mutation at the endogenous locus. However, a broad heterogeneity of mutations exists among individual patients with MPS I.Additionally, depending on the strength of the endogenous promoter, a large proportion of alleles may need to be edited to express therapeutic levels of the normal proteins. Due to the relative promoter strength of albumin as compared to the disease locus, editing only a small number of albumin alleles can lead to sufficient therapeutic protein expression. Further, by targeting a ‘safe harbor’ site, this strategy can be easily applied to other lysosomal diseases by using the same albumin-targeting cassette. Additionally, the risk of insertional mutagenesis is minimized through the use of a non-integrating virus for transgene delivery, and the precise targeting of a ‘safe harbor’ locus by nucleases, e.g., Cas9. In one embodiment, the albumin locus is selected for insertion of the promoterless IDUA coding region. With AAV carrying IDUA sequence and flanking homology sequences, IDUA sequence was inserted into albumin locus through homology-directed repair (HDR) or non-homologous end joining (NHEJ). The splicing donor sequence at exon 1 of albumin locus interacted with the splicing acceptor preceding the donor sequence. Therefore, under control of the endogenous albumin promoter, a fusion transcript of albumin exon 1 and IDUA was generated. Since exon 1 of albumin mainly encodes signal peptide and was cleaved thereafter, the mature protein was IDUA enzyme only. Then, IDUA enzymes are expressed by hepatocytes, secreted into plasma and endocytosed by cells from other tissues, achieving cross correction.

### Rationale for CRIPSR-Mediated Genome Editing to Treat MPS I Mice

The use of the CRISPR system having 2 vectors, e.g., 2 AAV vectors, may increase the rate of genome modification observed when more than 2 vectors are used because CRISPR, relative to other systems, has high targeting efficiency and ease of design. Moreover, higher dose (such as that needed when using 3 vectors) brings about higher rates of off-target effects, more challenge for vector production and higher manufacturing costs. With regard to the use of AAV, a Cas9 ortholog, Staphylococcus aureus Cas9 (SaCas9), is short enough to fit into AAV vectors (Ran et al., 2015). In this study, no off-target events were observed in the mice after AAV delivery of SaCas9 and guide RNAs. More interestingly, three independent gene therapy studies using SaCas9 observed undetectable (Yang et al., 2016) or minimal (Nelson et al., 2016; Tabebordbar et al., 2016) off-target effects, indicating a very high specificity. Considering the high efficiency and specificity, this SaCas9 system delivered by 2 AAV vectors was used. In one embodiment, the CRISPR/Cas system included one AAV vector encoding SaCas9 and guide RNA, and another encoding promoterless donor sequence. Assuming similar doses, AAV transduction and nuclease targeting efficiency, the efficiency of successful genome editing by CRISPR is expected to be higher. The CRISPR-mediated genome editing strategy allows for the use of a lower dose of AAV vectors for treating diseases, which brings minimized risk, ease of vector production and less expense.

A CRISPR-mediated in vivo genome editing strategy can treat both neurological and systemic diseases, e.g., MPS I. This CRISPR-mediated genome editing strategy can minimize the risk of insertional mutagenesis of lentiviral or retroviral vectors, and provide long-term therapeutic benefits which may not be provided by episomal vectors. Further, it has the potential to bring minimized safety risk, ease of vector production and less manufacturing expense by reducing the vector dose required for genome editing relative to other systems. This strategy can be utilized to treat a broad array of diseases including lysosomal diseases.

The status quo as it pertains to treating both systemic and neurological diseases of MPS I with minimized risk can be summarized as: little or none. it has been the case despite numerous and various approaches that have been taken. In clinical practice, ERT and HSCT have been used for MPS I patients, and provided significant therapeutic benefits. However, ERT failed to achieve neurological benefits, and it requires life-long, expensive treatments (Wraith et al., 2004). HSCT is associated with severe morbidity and mortality, while recipients continue to exhibit below normal IQ and impaired neurocognitive capability (Zielger et al., 2007). Gene therapy protocols with retroviral (e.g., Traas et al., 2007), lentiviral (e.g., Di Domenico et al., 2006) or AAV (e.g., Hinderer et al., 2015) vectors have been applied to treat MPS I in preclinical animal studies (mice or dogs). More recently, our study showed that ZFN-mediated in vivo genome editing can treat both systemic and neurological diseases of MPS I, resulting in pre-IND approval and advancement into human clinical trials. Nevertheless, the long-term efficacy of these approaches can be further improved, while the risk should be minimized.

The CRISPR genome editing approach can edit hepatocytes to provide sustained and substantial lysosomal enzyme, and efficiently treat both neurological and systemic diseases through cross-correction. By virtue of site-specific targeting of the albumin safe harbor locus, the risk of insertional mutagenesis is expected to be significantly reduced. Therapeutic horizons that have previously been unattainable through other treatment protocols will become attainable. It is also probable that advances made with CRISPR-mediated genome editing for treating MPS I disease will be transposable to other lysosomal diseases or monogenic diseases.

### AAV Delivery of CRISPR/Cas9 System for Genome Editing in Neonatal MPS I Mice

The CRIPSR system delivered by AAV vectors can edit hepatocytes to provide sustained and substantial IDUA enzyme to treat both systemic and neurological diseases in neonatal MPS I mice. AAV vectors carrying the CRISPR system are administered to, e.g., neonates, the primary treatment outcomes (IDUA expression, GAG reduction and cognitive abilities) are measured, safety profiles are monitored (clinical observations, histopathology, immune response) and gene editing events determined.

The design for CRISPR-mediated genome editing employs SaCas9 and guide RNA to mediate the insertion of cDNA, e.g., HEXB cDNA, into albumin locus and achieve expression of Hex enzyme. AAV8 vectors are liver-tropic, and SaCas9 is under control of a liver-specific promoter. By virtue of this, genome editing and transgene expression can be limited to hepatocytes. Systemic therapeutic benefits will be achieved through a phenomenon called ‘cross correction’ (Sands 2006). A total of four guide RNAs (gRNAs) were designed and transfected into fibroblast cells together with SaCas9. The ability of these gRNAs to guide SaCas9-mediated cleavage at the albumin locus was evaluated via the SURVERYOR assay. The results showed that one of the gRNAs, g1 (5′GTATCTTTGATGACAATAATGGGGGAT3′; SE#Q ID NO:3) mediated targeted DNA cleavage with the highest efficiency (11% indels, and was selected for future studies). Plasmids encoding SaCas9 and IDUA cDNA donor in MPS I mice through hydrodynamic injection. Only the mice receiving both plasmids had significant higher IDUA enzyme activities in liver (2.7 fold of wildtype levels). Mice receiving the plasmid encoding promoterless cDNA donor had no increase in IDUA activities. These results strongly support the feasibility of this CRISPR-mediated safe harbor genome editing strategy in treating MPS I mice.

in addition, because the GAG assay may not be sensitive enough to distinguish the small difference in brain GAG levels between adult MPS I and normal mice, HPLC-MS/MS was employed and significant increases in heparan sulfate and dermatan sulfate in MPS I brain tissues were identified. Additionally, HPLC-MS/MS identified significant increase in secondary storage materials of GM2 and GM3 gangliosides in MPS I mice brain. Therefore, we plan to quantify heparan sulfate, dermatan sulfate and gangliosides with HPLC-MS/MS for the main outcome measurement of storage materials in the brain.

### 1, Construct Design, Vector Production and Animal Injection

AAV8 vectors will be produced at University of Florida Vector Core, which has extensive experience in providing high quality AAV vectors for preclinical studies. Neonatal MPS I mice will receive co-delivery of AAV8-SaCas9 and AAV8-IDUA through temporal facial (percutaneous) vein. The injection will be conducted steadily and slowly (>15 seconds) to avoid potential hydrodynamic injection effects. Group assignment and dosage is listed in Table 1. To determine the optimal ratio between AAV8-SaCas9 and AAV8-IDUA, we will include two groups of mice receiving co-delivery of AAV vectors (1:5 or 1:10). In addition, we will add another group of mice receiving only AAV8-IDUA as a control. After weaning, we will conduct biweekly blood and urine collection. After 5 months post-dosing, we will euthanize all mice and harvest tissues including brain, heart, lung, liver, skeletal muscle, gonad and spleen.

TABLE 1

Group assignment of neonatal gene therapy

| Genotype | B | AAV8-CAS8 (vg/g body weight) | AAV8-IDUA (vg/g body weight) |
|---|---|---|---|
| MPS \| (kiua-1-) | 12 | 3×10¹⁸ | 1.5×10¹¹ |
| MPS \| (idua-1-) | 12 | 1.5×10¹⁰ | 1.5×10¹¹ |
| MPS \| (idua-1-) | 12 | 0 | 1.5×10¹¹ |
| MPS \| (idua-1-) | 12 | 0 | 0 |
| Normal (idua-/+) | 12 | 0 | 0 |

### 2. Primary Treatment Outcome Measurements

IDUA expression and storage reduction IDUA enzyme activities in plasma and tissues are measured with a standardized IDUA enzyme assay protocol (Ou et al., 2014b). GAG levels in urine and tissues are measured using a Blyscan glycosaminoglycan assay kit as previously described (Ou et al., 2014a). HPLC-MS/MS is also employed to quantify heparan sulfate, dermatan sulfate and gangliosides as a main parameter for storage reduction in the brain. These experiments will constitute the main outcome measurements of treatment effects of this genome editing strategy.

Behavior tests Prior to euthanasia, a 6-day trial of Barnes maze test is conducted that evaluates spatial memory and learning abilities (Barnes, 1979). Unlike other behavior tests such as Morris water maze, Morris T maze and radial arm tests, the Barnes maze test does not employ strong stress-induced stimulus. Therefore, the Barnes maze test can minimize the confounding factors brought by stress (Harrison et al., 2006). Further, the experimental setting at University of Minnesota Behavior Core applies the EthoVision program (Noldus), which also records and analyzes physical parameters including distance moved and velocity of mice. Preliminary data showed significant difference in the latency to escape between MPS I and normal mice at 4 months old of age. No significant differences were found in the aforementioned physical parameters between these mice, indicating that physical limitations were unlikely to be a confounding factor. Admittedly, the Barnes maze test involves visual cues to guide the mice to find the escape hole, and there have been reports about corneal clouding and reduced retinal function in MPS I mice (Ohlemiller et al., 2000). To rule out this potential confounding factor, the fear test that evaluates the learning and memory abilities of mice is employed (Shoji et al., 2014). The fear test has minimal physical involvement, making it ideal for functioning as a supplement to the Barnes maze test. Therefore, prior to euthanasia, the Barnes maze test is conducted, immediately followed by the fear test. The Barnes maze test is before the fear test because stress stimulus in the fear test may be a confounding factor for the Barnes maze test. The results from the behavior test show the efficacy of this genome editing strategy in treating neurological diseases of MPS I.

Histological analysis Additionally, cellular vacuolation is the characteristic microscopic finding of lysosomes engorged with GAG in MPS I mice (Ohmi et al., 2003). Reduced vacuolation has been observed in liver, spinal cord, heart, skeletal muscle, bone and joint of treated mice. Therefore, to determine potential therapeutic benefits, we will also evaluate the cellular vacuolation in these tissues.

### 3. Gene Editing and Vector Biodistribution Analysis

Gene modification analysis To assess the cutting efficiency of SaCas9, %indels at the albumin locus are measured in liver, spleen, brain as well as gonad (for monitoring germline transmission risk). A list of top 17 off-target sites was generated through a CRISPR/Cas9 target online predictor (Stemmer et al., 2015). To assess the specificity of SaCas9, %indels at in silico predicted off-target sites are measured in liver samples. Further, the ratio between fusion transcripts and total transcripts from albumin locus are measured by qRT-PCR, and PCR conducted to validate genome targeting at DNA level. Two sets of primers have been designed to detect inserted sequence at the albumin locus. Based on the size of the amplicons, we can determine the presence of insertion and the mechanism of insertion by PCR.

Biodistribution analysis qPCR is employed to determine AAV vector copy number in liver, spleen, brain, muscle, heart, lung and gonads. One safety concern about CRISPR gene therapy is that sustained transgene expression of SaCas9 will lead to immune responses or genome toxicity. Therefore, SaCas9 mRNA levels are evaluated by qRT-PCR and protein levels by Western blot as previously described (Yang et al., 2016). The AAV vector copy number and SaCas9 levels in gonads will be useful for assessing germline transmission risk.

### 4. Safety Profile

Clinical observation Mice are weighed biweekly after weaning, and mortality and morbidity events are noted on a daily basis. Additionally, organ weights of liver and spleen are measured when mice are euthanized. Preliminary data showed that the organ weights of spleen normalized by body weights in MPS I mice were significantly higher than those of normal mice. It indicates that MPS I mice recapitulate splenomegaly, one of the main symptoms of MPS I human patients. Therefore, it will be also interesting to see the effects of treatment on preventing organomegaly.

Humoral immune response The humoral immune response against IDUA proteins is measured by conducting ELISA of blood samples as described previously (Ou et al., 2014a). Similarly, an ELISA protocol (Ito et al., 2009) is used to detect neutralizing antibodies against the AAV8 capsid. Additionally, plasma IDUA levels can be a supplementary parameter: gradual decrease of plasma IDUA levels indicates immune response against transgene expression. These experiments will be a good measure of immune tolerance in neonatal gene therapy.

Assessment of tumor risk All tissues harvested during necropsy, as well as any mice found dead or euthanized due to moribund status, will be fixed in formalin. Then, the fixed tissues will be processed to slides for H&E staining, and evaluated by a board-certified pathologist. In addition, we will collect tumor tissues (if any) during necropsy and analyze the insertion sites as previously described (Walia et al., 2015). The profiling of insertion sites will be useful for determining the cause of tumor and designing corresponding methods to minimize the risk.

Statistical and gender consideration: For most experiments, results that are statistically significant when at least 6 mice are analyzed, will be considered as clinically significant. In anticipation of potential gender difference, 6 male and 6 female mice are in each group. It has been shown that gender influences liver transduction efficiency of AAV vectors through an androgen-dependent pathway (Davidoff et al., 2003). However, there was no difference in GAG levels between treated male and female mice because a small amount of IDUA is sufficient to reduce GAG storage. Therefore, a substantial gender difference in therapeutic benefits is not expected. Additionally, another potential gender difference could be performances in Barnes maze. As shown an earlier study, regardless of treatment, female mice spent significantly less time to find the escape hole. Therefore, when analyzing Barnes maze data, male and female mice are separately compared.

### Results

in mice receiving co-delivery of AAV8-SaCas9 and AAV8-IDUA, supraphysiological IDUA levels and efficient reduction of storage materials (GAG and gangliosides) are observed. Deep sequencing analysis will show high % indels in liver and undetectable in spleen, showing the high cutting efficiency limited in liver by liver-tropic vectors and liver-specific promoters. Besides, there will be a magnitude lower % indels in potential off-target sites. These results demonstrate the cutting efficiency and specificity of SaCas9. Additionally, SaCas9 levels and vector copy number are minimal, indicating elimination of vector genomes during the rapid proliferation of newborn liver. Based on transgene expression levels, the ratio between AAV8-SaCas9 and AAV8-IDUA is determined. Further, based on previous experience with neonatal lentiviral gene therapy (Ou et al., 2016), there will be no or low humoral immune response. Both male and female treated mice show significant better performances in behavior tests, indicating achieving neurological benefits. Histological analysis shows significant reduced cellular vacuolation in a variety of tissues including the CNS. More importantly, no cases of tumor formation are observed. Since incidence of tumor is influenced by promoter choice in the AAV vector (Chandler et al., 2015), and the donor construct is promoterless, which makes the tumor risk unlikely.

In mice treated with only AAV8-IDUA, there will be no IDUA transgene expression because this vector encodes a promoterless IDUA cDNA sequence. There is a remote possibility that minimal IDUA transgene expression is observed when AAV integrates the IDUA sequence in a vicinity of a promoter. Considering the low frequency of AAV random integration (Kaeppel et al., 2013), IDUA transgene expression from this mechanism will be minimal or undetectable.

Potential donor vector doses of 6x10¹⁰ vg/g body weight and up to at least 5x10¹¹ vg/g body weight, e.g., in neonatal mice (Yang et al., 2016) may be employed. The relative strength of the albumin promoter versus the endogenous OTC promoter enables a lower dose (1.5x10¹¹ vg/g of the donor vector). For higher expression, doses starting from 3x10¹¹ vg/mouse AAV8-IDUA may be employed. Any route of administration may be employed, e.g., vein injection (<50 µL for mice or i.p. injection, which is a routine substitute for i.v. injection.

### The Effects of in Utero Genome Editing Mediated by the CRISPR System

### Experiments

1. In utero injection The route of administration and gestational age of fetus are essential for the survival and transduction efficiency of IUGT. Preliminary data showed significant GAG accumulation on E14. Further, gene transfer at earlier time-points will result in more efficient transduction, probably due to the gradually reduced accessibility of stem cells (Endo et al., 2010). However, this study also showed that the survival rate of injected fetuses was directly correlated with gestational age: the later the injection was, the higher survival rate was. These results indicate that there is a balance between transduction efficiency and survival rate. Another factor to consider is targeting the albumin locus of hepatocytes. Therefore, intrahepatic injection should achieve the liver transduction. importantly, albumin expression is seen as early as E6 in mouse embryos (Trojan et al., 1995). The liver bud of mouse embryo forms at E9 and undergoes an accelerated growth between E10 and E15 (Medlock et al., 1983). Further, one study with intra-hepatic injection at E15 has achieved 93% survival rate and efficient transduction primarily in the liver (Lipshutz et al., 1999). Considering all the facts discussed above, intra-hepatic injection at E14 ensures high survival rate and efficient liver transduction. Therefore, time-dated pregnant mice at selected postcoital day are anesthetized by isoflurane inhalation. Then, the same dual AAV system is injected into the fetal liver through a transuterine approach as described previously (Lipshutz et al.,1999). Group assignment and dosage is listed in Table 2. According to Lipshutz et al, the injection volume is 5 µL in total. Based on the weight information of embryos (Mu et al., 2008), the virus titer is at least 1.2x10¹³ vg/mL. Notably, an extra group of mice injected with normal saline is the injection procedure control. Pups will not be manipulated before weaning.

TABLE 2

Group assignment of in utero gene therapy, * indicates that group of mice will be injected with normal saline as the injection control

| Genotype | n | AAV6-SaCas9 (vg/g body weight) | AAV8-IDUA (vg/g body weight) |
|---|---|---|---|
| MPS I(idua-l-) | 12 | 3×10¹⁰ or 1.5×10¹⁰ | 1.5×10¹¹ |
| MPS I (idua-l-) | 12 | 0 | 1.5×10¹¹ |
| MPS I (idua-l-) | 12 | 0 | 0 |
| MPS I (idua-l-)* | 12 | 0 | 0 |
| Normal (idua-l-*) | 12 | 0 | 0 |

2. Assessment of treated pups After weaning, biweekly blood and urine collection from the treated and control littermates is conducted. After 4 months post-dosing, all mice are euthanized and tissues including cerebrum, cerebellum, heart, liver, skeletal muscle, spleen and gonads are harvested. Then, all mice are comparatively evaluated (clinical observations, IDUA activities, storage reduction, behavior tests, histopathological analysis, biodistribution analysis, immune response and gene modification analysis). Mortality and morbidity events of injected fetuses is alos monitored. These experiments demonstrate the safety and efficacy of IUGT for treating MPS I, and provide important information for designing a clinical protocol of IUGT.

3. Comparison with neonatal gene therapy The same vector dose and experimental settings as the neonatal gene therapy are employed, which will enable a comparison of therapeutic benefits provided between two strategies.

4. Safety Profiling of mother mice A previous study has shown that AAV vectors may cross the placenta (Mattar et al., 2011). It is likely that some injected AAV vectors can cross the placenta and reach the tissues of the mother mice. Therefore, mortality and morbidity of these mother mice is monitored. After weaning, the mother mice are euthanized, SaCas9 mRNA levels are measured by qPCR and %indels in liver determined. The tissues including liver and injected site undergo histopathological analysis to evaluate potential pathology. Results from the mother mice are the first assessment of effects of IUGT on mothers.

As to the treated pups, we high survival rate (>90%) and normal organ development is expected. Further, compared with the control group, sustained supranormal IDUA activities and significant GAG storage reduction is observed. Deep sequencing shows efficient genome editing restricted in liver tissues, with minimal, if any, off-target effects. AAV vector copy number, SaCas9 mRNA and protein levels are undetectable due to the robust liver growth. Moreover, behavior tests (Barnes maze and fear test) show significantly better performance in treated MPS I mice compared with untreated MPS I mice. Considering the fact that the fetus has a naive immune system, immune tolerance of the transgene and vectors is expected. These results demonstrate the safety and efficacy of IUGT in treating both systemic and neurological diseases of MPS I.

As to the mother mice that receive in utero gene therapy, no morbidity or mortality is expected due to the procedure. A very low %indel at the albumin locus in liver tissues may be observed, and SaCas9 mRNA levels are undetectable. Histopathological analysis identifies no injection-associated pathology.

By employing the same technique and injection experimental setting as Lipshutz et al, similar survival rates (93%) are observed. If significantly lower survival rate (<80%) are observed, injections at E15-E18 are used because injections at late gestational age significantly improved the survival rate (Endo et al., 2010).

Besides determining insertional mutagenesis, germline transmission and effects on organ development, this study determines the extent to which the immature BBB and naive immune system in a fetus improves the efficacy of gene therapy.

### AAV Delivery of CRISPR/Cas9 System for Genome Editing in Adult MPS I Mice

Several studies (e.g., Ou et al., 2014a) showed that a consistent high level of IDUA in circulation could facilitate entry of IDUA into the CNS and improved performances of mice in behavior tests. Hydrodynamic tail vein injection of a plasmid encoding IDUA sequence into MPS I mice was conducted (Table 3). To eliminate any transgene expression in the CNS, the IDUA expression in liver by was restricted using a liver-specific hybrid promoter. Two days after the injection, the mice were perfused and euthanized, and depletion of brain capillaries was conducted. Interestingly, a significantly increase in IDUA activity and GAG reduction in the brain of injected mice was observed. These results indicated that IDUA proteins were expressed in the liver, resulting in high blood IDUA levels and a small but sufficient amount of IDUA in the CNS.

TABLE 3

IDUA enzyme activities and GAG levels in the brain of MPS I mice after injection. Data are shown as mean ± standard errors

| | IDUA enzyme activity (nmol/h/mg protein) | GAG levels (µg GAG/mg protein) |
|---|---|---|
| Treated MPS I(n=3) | 0.33±0.13 | 16.4±0.9 |
| Control MPS I(n=4) | 0 | 23.112 |
| p value | 0.03 | 0.04 |

Untargeted metabolomics analysis of liver and brain of Sandhoff disease (SD) mice was conducted with reverse phase liquid chromatography (RPLC). Principle component analysis of the metabolites identified showed a significant difference between SD mice and controls, indicating profound functional metabolic disturbances. The altered metabolites identified (74 in brain and 155 in liver) can be evaluated as potential surrogate biomarkers for response to therapies in this study. Further, global proteomic profiling of MPS I mouse brain with 2D-PAGE and LS-MS/MS (Ou et al., 2017) was conducted. 47 dysregulated proteins were identified. More importantly, both approaches identified potential biomarkers for prognosis and outcome measures for response to therapies. In summary, metabolomics and proteomics profiling can determine to what extent the treatment can normalize the alterations, and identify surrogate biomarkers for assessing response to therapies for future studies.

TABLE 4

Group assignment of adult gene therapy

| Genotype | n | AAV6-SaCas9 (vg/mouse) | AAV8-IDUA (vg/mouse) |
|---|---|---|---|
| MPS I (idua-l-) | 12 | 1.5×10¹¹ | 1.2×10¹² |
| MPS I (idua-l-) | 12 | 0 | 1.2×10¹² |
| MPS I (idua-l-) | 12 | 0 | 0 |
| Normal (idua-l+) | 12 | 0 | 0 |

### Experiments

### 1. Vector Injection

Adult MPS I mice receive co-delivery of AAV8-SaCas9 and AAV8-IDUA vectors (group assignment and dose in Table 4). The adult MPS I mice are randomized into each group controlled for age and body weight. An immune tolerization strategy is employed by injecting IDUA proteins into mice. Briefly, all mice receive IDUA infusion (5.8 mg/kg body weight) starting from the first day of life and weekly thereafter till AAV injection. Dr.

### 2. Treatment Outcome Measurements and Safety Profiling

Biweekly blood and urine collection are conducted, and the mice euthanized 7 months post-dosing (when the mice are at 8-month old). Briefly, we clinical observations, behavior tests, histopathological analysis, biodistribution analysis, gene modification analysis, measurements of IDUA expression and storage reduction are performed. Immune response against IDUA proteins is assessed by ELISA, which will determine the effects of immune tolerization. Collectively, these results assess safety and efficacy of this genome editing protocol in adult MPS I mice.

### 3. Metabolomics and Proteomics Analysis

Metabolites in tissues from all groups of mice are quantified to determine whether the treatment will normalize some of these metabolites. Similarly, 2D-PAGE and LC-MS/MS are employed to analyze proteomics profiles of MPS I mice as previously described (Ou et al., 2017). The results determine the treatment effects on altered proteomics and metabolomics profiles in MPS I mice, and identify metabolites or proteins as surrogate biomarkers for response to therapies.

Similar to results in neonates, increased IDUA enzyme activities, reduced storage of GAG and gangliosides are observed. As a result of successful immune tolerization, no significant antibodies or gradual loss of IDUA activities in plasma is observed. Better performance of mice receiving co-delivery of AAV8-SaCas9 and AAV8-IDUA in behavior tests is achieved. In addition, we reduced cellular vacuolation in multiple tissues including the CNS is observed, further supporting the efficacy of this genome editing strategy in proving neurological benefits. Through deep sequencing, %indels at the albumin locus of the liver, but not off-target locus, is measured. In addition, indels are observed from liver samples, not other tissues especially gonad, ruling out the possibility of germline transmission. SaCas9 levels and AAV vector copy number in the liver are minimal, showing the dilution effects due to liver growth. In addition, radiology analysis shows that abnormality in femur width and bone mineral density in MPS I mice is improved by genome editing. The degree of neuroinflammation manifested by activation of microglia and macrophage is alleviated. These results demonstrate the safety and efficacy of CRISPR-mediated genome editing in treating adult MPS I mice.

As to metabolomics and proteomics profiling, a large subset of altered metabolites and proteins is observed, and thereby correction of the profound metabolomics and proteomics impairments. The metabolites and proteins that respond well to the treatment, can be potential biomarkers for response to therapies in future studies.

The results from these experiments will constitute the first assessment of CRISPR-mediated in vivo genome editing of hepatocytes to treat MPS I disease.

Similar studies to express Hex and beta-galactosidase in mouse models of human diseases showed that the CRISPR approach described herein is broadly applicable.

### Example 3

The GM2 gangliosidoses, including Sandhoff disease (SD) and Tay-Sachs disease (TSD), are genetic disorders causing severe neurological diseases and premature death. GM2 gangliosidoses result from deficiency of a lysosomal enzyme β-hexosaminidase (Hex) and subsequent accumulation of GM2 gangliosides. Genetic deficiency of HEXA, encoding the Hex α subunit, or HEXB, encoding the Hex β subunit, causes TSD and SD, respectively. Currently, there is no effective treatment for human GM2 gangliosidoses, with palliative measures being the current standard of care. Gene therapy, a promising strategy, is being investigated in animal models. However, major obstacles must still be overcome including: (1) continuous, rather than pulsatile, delivery; (2) sufficient transgene product to the brain; (3) minimizing the vector-associated risk; and (4) timely therapeutic intervention prior to onset of irreversible damage. Therefore, there is a critical need to develop an innovative gene therapy protocol which surmounts these problems for treating GM2 gangliosidoses.

The CRISPR (Clustered Regulatory Interspaced Short Palindromic Repeats) system emerges as a powerful alternative with its high targeting efficiency and ease of design. Recently, a modified human Hex µ subunit (HEXM), incorporating sequence of both α and β subunits by forming a homodimer to degrade GM2 gangliosides (Karumuthil-Melethil et al., 2016), has been shown to able to treat both SD and TSD (Osmon et al., 2016; Tropak et al., 2016). Therefore, neonatal SD mice are injected with a dual AAV system (AAV8-SaCas9 and AAV8-HEXM-sgRNA), and a series of analyses are performed to assess the treatment efficacy.

### Materials and Methods

### Animals and Injections

SD mice (hexb-/-), purchased from the Jackson Laboratory, were generated by inserting a neomycin resistance cassette into exon 13 of the HEXB gene on the 129S4/SvJae background (Sango et al., 1995). SD mice (hexb-/-) and control mice were genotyped by PCR. All mouse care and handling procedures were in compliance with the rules of the Institutional Animal Care and Use Committee (IACUC) of the University of Minnesota.

Neonatal mice were injected with AAV vectors (<30 µL) through temporal facial vein on Day 1 or 2. Hydrodynamic injections of plasmids were performed in adult SD mice as described in Aronovich et al. (2013)

### Construct Design and in Vitro Confirmation

Four guide RNAs (gRNAs) were designed based on the locations to the insertion site and their off-target profiles. Then, these gRNAs were cloned into the pX602-AAV-TBG saCas9 plasmid. Each plasmid was transfected into mouse embryonic fibroblast (MEF) cells, and cells were subsequently harvested for PCR amplification. In order to determine the gRNA cleavage activity of the gRNA constructs, an in vitro SURVEYOR assay was performed on the PCR product (SURVEYOR mutation detection kit, Transgenomic Inc., cat#: 706020).

### Vector Production

AAV-HEXM-gRNA and AAV-SaCas9 were packaged into AAV8 vectors at the Children’s Hospital of Philadelphia Research Vector Core. The titer was verified by SDS PAGE and silver staining. The core follows Good Laboratory Practice (GLP) guidelines.

### Depletion of Brain Capillaries

To rule out the possibility that enzyme activities in the brain come from capillary cells and blood, all mice were transcardially perfused with 35 mL PBS, and depletion of brain capillaries was performed as described in WNG ET AL. (2013).

### Hex Enzyme Assay

Tissues were homogenized and protein concentrations were measured as described in Ou et al (2016).. Hex A and Hex total enzyme activities in plasma and tissues were measured using a previously described enzyme assay protocol (Bradbury et al. (2013). 4-Methylumbelliferyl N-acetyl-b-D-glucosaminide (4MUG, Sigma # M2133) and 4-Methylumbelliferyl-6-sulfa-2-Acetoamido-2-Deoxy-beta-D-Glucopyranoside Potassium salt (4MUGS, TRC # M335000) were used for measuring Hex total and Hex A activities, respectively.

### Ganglioside Quantification

GM2 gangliosides were quantified using HPLC-MS/MS as described in Pryzbilla et al. (2018).. The mouse brain (1 g wet tissue/6 mL CHAPS solution), heart (1 g wet tissue/6 mL CHAPS solution), liver (1 g wet tissue/6 mL CHAPS solution), and spleen (1 g wet tissue/6 mL CHAPS solution) samples were homogenized in 2% CHAPS solution. Protein precipitation with 200 µL of methanol was performed to extract gangliosides GM2 from 50 µL of homogenate in the presence of internal standards (d3-GM2(18:0)). The 10% study sample extracts from each tissue type were pooled to prepare a quality control (QC) sample for that tissue. The QC samples were injected every 5 study samples to monitor the instrument performance. Sample analysis was performed with a Shimadzu 20AD HPLC system, coupled to a 6500QTRAP mass spectrometer operated in positive MRM mode. Data processing was conducted with Analyst 1.5.2 (Applied Biosystems). The relative quantification of lipids is provided, and the data were reported as the peak area ratios of the analytes to the corresponding internal standards. The relative quantification data generated in the same batch are appropriate to compare the change of an analyte in a test sample relative to other samples (e.g., control vs. treated, or samples in a time-course study). The coefficient variances (CV) of gangliosides in QC samples are provided. The ganglioside species with CV greater than 15% in QC sample are highlighted in yellow, and these results should be interpreted with caution.

### Behavior Tests

The pole test was performed as described in Ogawa et al. (1985). Rotarod analysis was performed using an adapted protocol described in Hockey et al. (2003). Fear conditioning was performed according to an established protocol (Martin-Fernandez et al., 2017). All three behavior tests were performed at the Mouse Behavior Core, University of Minnesota.

### Histology and Immunohistochemistry

After perfusion and fixation in 10% neutral buffered formalin, tissues were processed into paraffin using standard histology techniques, sectioned at a thickness of 4 µm , stained with hematoxylin and eosin (H&E), and evaluated by light microscopy.

For Hex A immunohistochemistry (IHC) preparations, 4 µm formalin-fixed, paraffin-embedded sections of tissue were deparaffinized, rehydrated, and subjected to heat-induced antigen retrieval (using 10 mM Citrate buffer pH 6.0) in a steamer prior to performing the IHC procedure on a Dako Autostainer. IHC for Hex A was performed using a rabbit anti-Hex A polyclonal antibody (Thermo Fisher Scientific # PA5-45175) as primary antibody. Detection was achieved using a rabbit EnVision™+ Kit (catalog K4011, Dako) with DAB as the chromogen. All work was done at the University of Minnesota Masonic Cancer Center Comparative Pathology Laboratory.

### Results

### Construct Design and Verification

SaCas9 and guide RNA mediate the insertion of promoterless cDNA donor into albumin locus and achieve expression of Hex enzyme. AAV8 vectors are liver-tropic, and SaCas9 is under control of a liver-specific promoter. By virtue of this, genome editing and transgene expression can be limited to hepatocytes. Systemic therapeutic benefits are expected to be achieved through a phenomenon called ‘cross correction’ (Sands et al., 2006). A total of four guide RNAs (gRNAs) were transfected into mouse embryonic fibroblast cells together with SaCas9.

5′GTATCTTTGATGACAATAATGGGGGAT3′ (SEQ ID NO:4)
5′GGCAGAATGACTCAAATTACGTTGGAT3′ (SEQ ID NO:5)
5′TTCAACTGTATCCAACGTAATTTGAGT3′ (SEQ ID NO:6)
5′GATCGGGAACTGGCATCTTCAGGGAGT3′ (SEQ ID NO:7)

The ability of these gRNAs to guide SaCas9-mediated cleavage at the albumin locus and to promote DNA double strand break was evaluated via the SURVERYOR assay. The results showed that one of the gRNAs, g1 (5′GTATCTTTGATGACAATAATGGGGGAT3′; SEQ ID NO:3) mediated targeted DNA cleavage with the highest efficiency (11% indels), and was selected for the following studies.

In addition, the plasmids encoding SaCas9 and HEXB cDNA donor were tested in adult SD mice through hydrodynamic injection. Only the mice receiving both plasmids had significant higher Hex total activities in the liver (45% of wildtype levels). Notably, there is no significant increase in Hex A (αα) activities, indicating that the increase of Hex total activities mainly comes from Hex B (ββ) through transgene expression of HEXB cDNA. Mice receiving the plasmid encoding promoterless cDNA donor showed no increase in Hex A or Hex total activities. These results strongly support the feasibility of this CRISPR-mediated ‘safe harbor’ genome editing strategy in treating SD mice.

### Study With Hydrodynamic Injections

Since GM2 gangliosidoses are primarily neurological diseases, previous gene therapy studies focused on direct injection into the brain. This liver-targeting gene editing strategy is based on previous studies in MPS I mice. These studies, together with other studies in MPS II mice (Laoharawee et al., 2018; Cho et al., 2015), MPS IIIA mice (Rozaklis et al., 2011), MPS VII mice (Vogler et al., 2005), Krabbe mice (Lee et al., 2005), metachromatic leukodystrophy mice (Matzner et al., 2005), α-Mannosidosis mice (Blanz et al., 2008), and α-Manosidosis pig (Crawley et al., 2006), showed that when a constant supply of enzyme is present in the bloodstream at high levels, a small amount may be able to cross the BBB into the central nervous system.

To further support this, hydrodynamic injection of a plasmid encoding HEXM sequence into adult SD mice was performed. To eliminate any transgene expression in the CNS, the HEXM expression was restricted in the liver by using a liver-specific promoter/enhancer (the human α-1-antitrypsin [hAAT| promoter and human apolipoprotein [ApoE] enhancer). Two days after the injection, the mice were transcardially perfused, and depletion of brain capillaries was performed. interestingly, a significant increase in Hex A and Hex total activities were observed in the brain of injected mice. These results indicated that Hex proteins were expressed in the liver, resulting in high blood Hex enzyme levels and a small, but sufficient, amount of Hex enzyme in the CNS. In addition, the fact that both Hex A and Hex total activities increased support the therapeutic potential of the HEXM sequence (below is an alignment of the sequences of Hex A and HexM, and the sequence of HexM) in treating both TSD and SD.

[symbol]

Met Thr Ser Ser Arg Leu Trp Phe Ser Leu Leu Leu Al
a Ala Ala Phe Ala Gly Arg Ala Thr Ala Leu Trp Pro
Trp Pro Gln Asn Phe Gln Thr Ser Asp Gln Arg Tyr Va
l Leu Tyr Pro Asn Asn Phe Gln Phe Gln Tyr Asp Val
Ser Ser Ala Ala Gln Pro Gly Cys Ser Val Leu Asp Gl
u Ala Phe Gln Arg Tyr Arg Asp Leu Leu Phe Gly Ser
Gly Ser Trp Pro Arg Pro Tyr Leu Thr Gly Lys Arg Hi
s Thr Leu Glu Lys Asn Val Leu Val Val Ser Val Val
Thr Pro Gly Cys Asn Gln Leu Pro Thr Leu Glu Ser Va
l Glu Asn Tyr Thr Leu Thr Ile Asn Asp Asp Gln Cys
Leu Leu Leu Ser Glu Thr Val Trp Gly Ala Leu Arg Gl
y Leu Glu Thr Phe Ser Gln Leu Val Trp Lys Ser Ala
Glu GlyThr Phe Phe Ile Asn Lys Thr Glu Ile Glu Asp
Phe Pro Arg Phe Pro His Arg Gly Leu Leu Leu Asp T
hr Ser Arg His Tyr Leu Pro Leu Lys Ser Ile Leu Asp
Thr Leu Asp Val Met Ala Tyr Asn Lys Leu Asn Val P
he His Trp His Leu Val Asp Asp Gln Ser Phe Pro Tyr
Glu Ser Phe Thr Phe Pro Glu Leu Met Arg Lys Gly S
er Tyr Ser Leu Ser His IleTyr Thr Ala Gln Asp Val
Lys Glu Val Ile Glu Tyr Ala Arg Leu Arg Gly Ile Ar
g Val Leu Ala Glu Phe Asp Thr Pro Gly His Thr Leu
Ser Trp Gly Pro Gly Ile Pro Gly Leu Leu Thr Pro Cy
s Tyr Ser Gly Ser Glu Pro Ser Gly Thr Phe Gly Pro
Val Asn Pro Ser Leu Asn Asn Thr Tyr Glu Phe Met Se
r Thr Phe Phe Leu Glu Val Ser Ser Val Phe Pro Asp
Phe Tyr Leu His Leu Gly Gly Asp Glu Val Asp Phe Th
r Cys Trp Lys Ser Asn Pro Glu Ile Gln Asp Phe Met
Arg Lys Lys Gly Phe Gly Glu Asp Phe Lys Gln Leu Gl
u Ser Phe Tyr Ile Gln Thr Leu Leu Asp Ile Val Ser
Ser Tyr Gly Lys Gly Tyr Val Val Trp Gln Glu Val Ph
e Asp Asn Lys Val Lys Ile Gln Pro Asp Thr Ile Ile
Gln Val Trp Arg Glu Asp Ile Pro Val Asn Tyr Met Ly
s Glu Leu Glu Leu Val Thr Lys Ala Gly Phe Arg Ala
Leu Leu Ser Ala Pro Trp Tyr Leu Asn Arg Ile Ser Ty
r Gly Gln Asp Trp Arg Lys Phe Tyr Lys Val Glu Pro
Leu Ala Phe Glu GlyThr Pro Glu Gln Lys Ala Leu Val
Ile Gly Gly Glu Ala Cys Met Trp Gly Glu Tyr Val A
sp Ala Thr Asn Leu Val Pro Arg Leu Trp Pro Arg Ala
Gly Ala Val Ala Glu Arg Leu Trp Ser Asn Lys Leu T
hr Arg Asp Met Asp Asp Ala Tyr Asp Arg Leu Ser His
Phe Arg Cys Glu Leu Val Arg Arg Gly Val Ala Ala G
in Pro Leu Tyr Ala Gly Tyr Cys Asn Gln Glu Phe Glu
Gln Thr (SEQ ID NO:10)

(WO 2015/150922, the disclosure of which is incorporated by reference herein)

### AAV Delivery of the Gene Editing System to Treat SDw

### Hex Enzyme Activities

Neonatal SD mice (n=10) received co-delivery of AAV8-SaCas9 (5x10⁹ vg/g body weight) and AAV8-HEXM-gRNA (3×10¹⁰ vg/g body weight) through temporal facial vein. A group of SD mice receiving the donor only (AAV8-HEXM-gRNA, n=4) was also included as controls. Plasma Hex A and Hex total activities in Cas9+donor treated SD mice increased markedly up to 144 and 17 fold of wildtype levels, respectively. In mice treated with the donor alone, the Hex enzyme activities did not significantly increase, indicating that there was no episomal transgene expression from the promoterless donor. After 4 months, all mice were euthanized and tissues were harvested for further analyses. Hex A activities in the liver, heart and spleen increased to 25, 3 and 2 fold of wildtype levels, respectively. Hex total activities in the liver, heart and spleen increased 7 fold, 120% and 79% of wildtype levels, respectively. More interestingly, Hex A and Hex total activities in the brain of Cas9+donor treated mice also increased significantly (compared with untreated SD mice, p<0.05).

### GM2 Gangliosides

Further, HPLC-MS/MS was applied to quantify the GM2 gangliosides in tissues., GM2 gangliosides were significantly reduced in the liver, heart and spleen (p<0.05). However, the total GM2 gangliosides in the brain were not significantly reduced in the Cas9+donor treated mice.

### Histopathology and Immunohistochemistry

Cellular vacuolation is the characteristic microscopic finding of lysosomes engorged with storage materials in the murine model of lysosomal diseases. To assess whether the treatment can reduce cellular vacuolation, histological analysis of the brain and liver was performed. Untreated SD mice showed the typical hepatic and cerebral lesions associated with lysosomal accumulation: Kupffer cell and neuronal cell hypertrophy and vacuolation (with small, well defined vesicles of variable sizes, with clear to pale-eosinophilic content). Moreover, within the brain, the lysosomal accumulation (manifested as cellular vacuolation) is present in variable degrees in all the main anatomic areas (brain cortex, hippocampus, thalamus, hypothalamus, pons and cerebellum). In contrast, there is an absence of Kupffer cell vacuolation in treated SD mice, with the morphology of the liver being comparable from this perspective to normal mice. More importantly, the neuronal lysosomal accumulation was reduced in most treated SD mice.

To test whether the enzyme expressed from the liver can enter the CNS, immunostaining against Hex A was performed with anti-Hex A polyclonal antibody, which has been shown to be able to target the µ subunit (expressed by HEXM construct) (Karumuthil-Melethil et al., 2017). There is an increased intensity of labelling that could be consistent with Hex enzyme in the brain of 1 out of 3 treated mice. These results further corroborate the findings of increased enzyme activities in the brain of treated mice.

### Behavior Tests

Three months post dosing, a battery of behavior tests was performed to assess the treatment efficacy. In the pole test (assessing bradykinesia) and the fear conditioning (assessing learning and memory), no significant differences were observed between untreated SD mice and normal mice. These results indicate that at least at this age, these two tests could not distinguish SD mice from normal mice. However, in the rotarod test, which assesses coordination, motor function and motor memory, a significant difference between untreated SD and normal mice were observed. Moreover, the Cas9+donor treated mice had significantly improved performance compared with untreated SD mice (p<0.05). These results indicate that this liver-targeting gene therapy achieved neurological benefits.

### Discussion

Although expressing only β subunit is expected to efficiently treat SD mice, the sialidase bypass does not exist in humans, making translation of this strategy into clinical practice difficult. Further, optimal production of HexA enzyme is suggested to be expressing both subunits because the overexpression of one subunit may rapidly deplete the pool of its endogenous subunit partner (Itakkura et al., 2006). Previous study (Cachon.Gonzalez et al., 2006) showed that co-expression of both subunits achieved higher HexA activities in SD mice or cats. However, it is difficult to package both HEXA and HEXB cDNA into one AAV vector, while the use of two vectors brings about higher manufacturing cost and vector-associated risk. To this end, a modified a subunit incorporating partial sequence of β-subunit was designed. This modified subunit (µ) can form a stable dimeric enzyme, HexM, which efficiently degrades GM2 in human cells as well as SD mice. Expression of HEXM is expected to achieve greater therapeutic benefits than that is achieved through expression of one subunit alone, which would result predominantly in the formation of either HexS (αα) or HexB (ββ). Another benefit for using this HEXM construct is the ability to treat both TSD and SD as shown in two studies. In this study, application of the HEXM construct successfully achieved significant Hex A and total activities, demonstrating its remarkable therapeutic potential.

There are no effective therapies for the GM2 gangliosidoses, with palliative measures being the current standard of care. Enzyme replacement therapy (ERT) (Tsuji et al., 2011), substrate reduction therapy (SRT) (Maegawa et al., 2009), chemical chaperone therapy (Osher et al., 2011) and bone marrow transplantation (BMT) (Jacobs et al., 2005), only achieve limited therapeutic benefit in animal models. Gene therapy holds promise for treating lysosomal diseases as it has potential for permanent, single-dose treatment. GM2 animal model studies include gene modification using lentiviral (Kyrkanides et al., 2005) and AAV vectors (Chachon-Gonzalez et al., 2012), but these methods have integration and persistence drawbacks. Integrating vectors, such as lentiviral vectors, randomly integrate into the genome, raising potential concerns of insertional mutagenesis (Hacein-Bey-Abina et al., 2003). Clinical trials treating X-linked severe combined immunodeficiency with retroviral gene therapy resulted in leukemia for 2 patients through oncogene activation by vector integration. Meanwhile, AAV, mainly an episomal vector, is not expected to provide permanent transgene expression. It was shown that transgene expression from episomal AAV vectors was rapidly lost after one round of cell division (Nakai et al., 2001). Unfortunately, secondary administration of AAV vectors often fails to rescue expression, due to the immune response to primary vector delivery (Calcedo et al., 2013). Collectively, treatment protocols providing sustained therapeutic benefits with minimized safety risks for patients with GM2 gangliosidoses are in desperate need.

In a previous study with ZFNs, the genome modification rate was relatively low. The low probability of all 3 AAV vectors transfecting the same cell explains this low efficiency modification rate. Although sufficient to treat MPS |mice, application of this strategy in human patients will require a large amount of high-titer AAV vectors. Higher dose brings about a greater rate of off-target effects, more challenging vector production and increased manufacturing costs. A new Cas9 ortholog, Staphylococcus aureus Cas9 (SaCas9), that fits into AAV vectors, has been discovered (Ran et al., 2015). In this study, no off-target events were observed in the mice after AAV delivery of SaCas9 and guide RNAs. More interestingly, three independent gene therapy studies using SaCas9 observed undetectable (Yang et al., 2016) or minimal (Tabebordbar et al., 2016; nelson t al., 2016) off-target effects, indicating very high specificity. Considering the high efficiency and specificity, we plan to utilize this SaCas9 system delivered by AAV vectors to optimize our previous strategy with ZFNs. As opposed to 3 AAV vectors used in the study with ZFNs, this CRISPR system only requires 2 vectors: one AAV vector encoding SaCas9, the other encoding the promoterless donor sequence and guide RNA. Assuming similar doses, AAV transduction and nuclease targeting efficiency, the efficiency of successful genome editing by CRISPR is expected to be higher than that mediated by ZFNs. The CRISPR-mediated genome editing strategy will enable us to use lower doses of AAV vector for treating lysosomal diseases, which brings minimized risk, ease of vector production and less expense.

Although the etiology is not fully understood, the GM2 gangliosidoses are primarily neurological disorders. Therefore, most previous gene therapy studies focused on direct injections into the brain. These approaches are of limited use due to several drawbacks: (1) highly invasive nature; (2) difficulty in achieving uniform and global distribution throughout the brain (Passini et al., 2002) (3) the inability to treat systemic diseases that become prominent when animals live longer because neurological diseases are treated; (4) genotoxicity due to overexpression of HexA in neurons (Golebiowski et al., 2017). Alternatively, fusing lysosomal enzyme with other proteins to target the CNS has also been tested (Ou et al., 2018), while the application into gangliosidoses has not yet accomplished. In this study, based on our previous experiences with intravenous administration, a liver-targeting genome editing strategy was assessed in treating SD mice. Admittedly, the fact that GM2 gangliosides levels were not significantly reduced in the brain seems confusing, However, rotarod analysis showed improvements in motor function of treated SD mice, and histological analysis showed reduced neuronal vacuolation. These results support that this liver-targeting gene therapy can achieve significant neurological benefits. Moreover, considering the dose used in this study is relatively low (3.5x10¹⁰ vg/g body weight), the treatment efficacy in the brain can be significantly improved by increasing the dose.

### REFERENCES

Aronovich et al., PLoS One, 8:e78161 (2013).
Blanz et al., Hum Mol Genet., 17:3437 (2008).
Bradbury et al., Mol. Ther., 21:1306 (2013).
Cachon-Gonzalezet al., Proc. Natl Acad. Sci. USA, 103:10373 (2006).
Cachon-Gonzalez et al., Mol. Ther, 20:1489 (2012).
Calcedo and Wilson, Front Immunol., 4:341 (2013).
Cho et al., Orphanet. J. Rare. Dis., 10:141 (2015).
Crawley et al., Mol. Genet. Metab., 89:48 (2006).
Golebiowski Hum. Gene Ther., 28:510 (2017).
Hacein-Bey-Abina et al., Science, 302:415 (2003).
Hockly et al., Brain Res. Bull., 61:469 (2003).
Itakura et al., Biol. Pharm. Bull., 29:1564 (2006).
Jacobs et al., Bone Marrow Transplant., 36:925 (2005).
Karumuthil-Melethil et al., Mol Ther., 25:S95 (2017).
Karumuthil-Melethil et al., Hum. Gene Ther., 27:509 (2016).
Kyrkanides et al., Brain Res. Mol. Brain Res., 133:286 (2005).
Laoharawee et al., Mol. Ther., 26:1127 (2018).
Lee et al., FASEB J. 19:1549 (2005).
Maegawa et al., Mol. Genet. Metab., 98:215 (2009).
Martin-Fernandez et al., Nat. Neurosci.. 20:1540 (2017).
Matzner et al., Hum. Mol. Genet., 14:1139 (2005).
Nakai et al., J. Virol., 75:6969 (2001).
Nelson et al., Science, 351:403 (2016).
Ogawa et al., Res. Commun. Chem. Pathol. Pharmacol., 50:435 (1985).
Osher et al., Mol. Genet. Metab., 102:356 (2011).
Osmon et al., Hum. Gene Ther., 27:497 (2016).
Ou et al., Mol. Ther., In press.
Ou et al., Mol. Genet. Metab., 111:116 (2014).
Ou et al., Mol. Genet. Metab, 123:105 (2018).
Ou et al., Mol. Genet. Metab. Rep., 8:93 (2016).
Ou et al., Mol. Genet. Metab., 1096:30437 (2018).
Passini et al., J. Neurosci., 22:6437 (2002).
Przybilla et al., Mol. Genet. Metab., Under review.
Ran et al., Nature, 520:186 (2015).
Rozaklis et al., Exp. Neurol., 230:123 (2011).
Sands and Davidson, Mol. Ther., 13:839 (2006).
Sango et al., Nat. Genet., 11:170 (1995).
Tabebordbar et al., Science, 351:407 (2016).
Tropak et al., Mol. Ther. Methods Clin. Dev., 3:15057 (2016).
Tsuji et al., Ann. Neurol., 69:691 (2011).
Vogler et al., Proc. Natl. Acad. Sci. USA, 102:14777 (2005).
Wang et al., Proc. Natl. Acad. Sci. USA, 2013 Feb 19;110(8):2999-3004.
Yang et al., Nat. Biotechnol., 34:334 (2016).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prevent, inhibit or treat a disease in a mammalian cell, comprising:
administering an effective amount of i) Cas or an isolated nucleic encoding Cas, and ii) isolated nucleic acid for one or more gRNAs comprising a targeting sequence for a genomic target and nucleic acid comprising a coding sequence for a prophylactic or therapeutic gene product flanked by homology arms, or an effective amount of iii) isolated nucleic encoding Cas and nucleic acid for one or more gRNAs comprising a targeting sequence for a genomic target, and iv) isolated nucleic acid comprising a coding sequence for a prophylactic or therapeutic gene product flanked by homology arms,
wherein the expression of the coding sequence in the mammal prevents, inhibits or treats the disease.

2. The method of claim 1 wherein the disease is mucopolysaccharidosis, a lysosomal storage disease, hemophilia, thalassemia, or sickle cell disease.

3. The method of claim 1, wherein the targeting sequence or homology arms are targeted to an intron.

4. The method of claim 1, wherein one or more adeno-associated virus (AAV), adenovirus or lentivirus is/are employed to deliver at least one of i) or ii) or at least one of iii) or iv).

5. The method of claim 4 wherein a first rAAV delivers nucleic acid encoding Cas.

6. The method of claim 5 wherein a second rAAV delivers the nucleic acid comprising the targeting sequence and the coding sequence.

7. The method of claim 5 wherein the first or second AAV is one of serotypes AAV1-9 or AAVrh10.

8. The method of claim 5 wherein the first and the second rAAVs are different serotypes.

9. The method of claim 1 wherein the mammal is a human.

10. The method of claim 1 wherein one or more of the gRNAs target an albumin locus, Rosa26 locus, BCR locus, AAVS1 locus, CCR5 locus, HPRT locus, or alpha fetoprotein locus.

11. The method of claim 1 wherein the disease is mucopolysaccharidosis type I, type II type III, type IV, type V, type VI or type VII.

12. The method of claim 1 wherein the disease is Tay-Sachs disease or Sandhoff disease (GM2-gangliosidosis disease).

13. The method of claim 1 wherein the coding sequence encodes iduronidase, beta-globin, iduronate, beta galactosidase, sulfatase, arylsulfatase B, hexM, hexoaminidase A or hexosaminidase B.

14. The method of claim 3 wherein the intron is an albumin gene intron.

15. The method of claim 3 wherein the intron is the first intron.

16. The method of claim 1 wherein the targeting sequence targets sequences within the first 500, 400, 300, 200, or 100 nucleotides of the intron.

17. The method of claim 1 wherein the Cas comprises Streptococcus pyogenes (SpCas9), Staphylococcus aureus (SaCas9), Streptococcus thermophilus (StCas9), Neisseria meningitidis (NmCas9), Francisella novicida (FnCas9),Campylobacter jejuni (CjCas9), CasX and CasY, Cas12a (Cpf1), Cas14a, eSpCas9, SpCas9-HF1, HypaCas9, Fokl-Fused dCas9, or xCas9.

18. The method of claim 1 wherein liposomes are employed to deliver i), ii), iii), iv), or any combination thereof.

19. The method of claim 1 wherein the nucleic acid comprising a coding sequence for a prophylactic or therapeutic gene product is not operably linked to a promoter.

20. A composition comprising a first vector comprising an isolated nucleic encoding Cas9 and a second vector comprising an isolated nucleic comprising sequences for one or more gRNAs comprising a selected targeting sequence and a selected coding sequence flanked by homology arms, or a first vector comprising an isolated nucleic encoding Cas9 and an isolated nucleic comprising sequences for one or more gRNAs comprising a selected targeting sequence and a second vector comprising a selected coding sequence flanked by homology arms.
